(19) United States
(12) Patent Application Publication
(10) Pub. No.: US 2010/0004171 A1
(43) Pub. Date: Jan. 7, 2010

(54) Therapeutic Methods for Neuropathic Pain

(76) Inventors: Nicolas G. Bazan, New Orleans, LA (US); Jian-Guo Cui, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/375,634

(22) PCT Filed: Aug. 8, 2007
(86) PCT No.: PCT/US07/75500

§ 371 Date: Aug. 10, 2009

Related U.S. Application Data
(60) Provisional application No. 60/934,938, filed on Aug. 8, 2006.

(51) Int. Cl. A61K 38/16; A61K 31/167; C12Q 1/02

(52) U.S. Cl. 514/12; 514/629; 435/29

(57) ABSTRACT

The agrin protein was shown to be important in preventing the development of neuropathic pain, as well as in treating neuropathic pain. Both agrin protein and gene expression were shown to be down-regulated in mammals with neuropathic pain. Increasing either agrin gene expression or protein resulted in a decrease in the development of neuropathic pain. Agrin protein or the C-terminal agrin fragments can be administered in a number of ways, preferably by intrathecal injection. In addition, agrin can be increased by administering a compound shown to affect agrin gene expression or agrin protein concentration, e.g., SCP-I and SCP-M1 (also known as JMM). Agrin protein decrease was shown to be prevented by administering an NMDA receptor antagonist, e.g., MK801. Agrin and a C-terminal agrin fragment also induced phosphorylation of the NMDA receptor subunit NR1 at the serine residue site which led to suppression of neuropathic pain.

The benefit of the 8 Aug. 2006 filing date of provisional application 60/934,938 (which was a conversion of U.S. nonprovisional application Ser. No. 11/500,722), now abandoned, is claimed under 35 U.S.C. § 119(e) in the U.S., and is claimed under applicable treaties and conventions in all countries.

This invention pertains to methods to prevent or ameliorate neuropathic pain by increasing the concentration of agrin or certain fragments of agrin in the central nervous system.

Neuropathic pain. Following an injury or disease of either nerve or peripheral tissue, a type of chronic pain called neuropathic pain (NP) may frequently develop, with ongoing, spontaneous, paroxysmal, and lancinating pain components. Such NP is almost invariably associated with abnormalities of cutaneous sensibility in the forms of allodynia (sensation of pain from stimuli that are not normally painful), hyperalgesia (increased sensation to normally painful stimuli), and dysesthesis (unpleasant abnormal sensation). Although knowledge about neuropathic pain mechanisms has advanced tremendously, satisfactory treatment options for NP have been elusive.^{[1,2]} The features of neuropathic pain are known to be different from that of the general, nociceptive type of pain. Nociceptive type of pain is a chronic or acute pain associated with a painful stimulus. Most animal models used to study pain and its treatment are based on the nociceptive type of pain, e.g., tail flick or hot plate models.^{[83]} Neuropathic pain can be induced by innocuous stimuli, and responds much less to some medications than does the nociceptive type. For example, opioids seldom have an analgesic effect on neuropathic pain, while opioids are successful in producing an analgesic effect on nociceptive pain.^{[82,83]} Neuropathic pain can result from peripheral nerve trauma (e.g., amputation), infection (e.g., post-herpetic neuralgia), infarct, or metabolic disturbance (e.g., diabetic neuralgia). New treatment strategies are needed for treatment of neuropathic pain.^{[84]}

NP development involves peripheral and central neuronal alterations that result in neuropathic hyperexcitability. In the peripheral tissues, ectopic discharges from injured nerves (or neuroma) input continuous excitations into the spinal dorsal horn, and then further to the brain, that can lead to long-term central hyperexcitability.^{[3]} Moreover, cross-talk structures between A-fibers (a myelinated nerve fiber that conducts non-nociceptive sensations, such as touch, warmth, and vibration), or between an A-fiber to a C-fiber (a non-myelinated nerve fiber that conducts nociceptive sensations, such as pain, burn), or sprouting from injured nerve fibers, adjacent nerve fibers, and sympathetic fibers, may also enhance excitatory inputs.^{[4-6]} In addition, sensitized cutaneous mechanoreceptors around damaged tissue, increased expression of receptors, and abnormality of ion channels can all contribute to hyperexcitability in the CNS.^{[7-9]}

On the other hand, up-regulation of excitatory neurotransmitters (e.g., glutamate, aspartate, substance P (SP), cholecystokinin, calcitonin gene-related peptide (CGRP), etc.) and down-regulation of inhibitory neurotransmitters (e.g., gamma-amino butyric acid (GABA), adenosine, galanin, etc.) in the spinal dorsal horn produce another basis for such central hyperexcitability.^{[10-13]} Glutamate and aspartate excite postsynaptic NMDA receptors, while SP, CGRP, and protein kinase C synergistically remove a magnesium block that normally limits NMDA receptor overexcitation. Overexcited NMDA receptors then contribute to long-term overreaction to innocuous or slight pain stimuli.^{[14-15]} Concomitantly, following an injury, the receptor field expands.^{[16, 17]} In addition, myelinated fibers sprouting from the terminals in lamina III and IV (where non-nociceptive inputs arrive) to lamina I and II (where nociceptive afferents terminate) may constitute a structural basis for hyperexcitability. Recent data also indicate that the inflammatory cells and mediators (e.g., macrophages, IL-1, -6, -10, TNF-alpha, COX-2)^{[18-23]} and growth factors (e.g., NGF, BDNF, GDNF, EGF, and neurotrophin-3) as well as related secondary intracellular signaling systems (e.g., protein kinases A and C, tyrosine kinase, mitogen-activated protein kinase, protein phosphorylation, nitric oxide, and P2X) are involved in neuropathic pain development.^{[24-28]}

Agrin and neurons in the CNS. Agrin is a heparin sulfate proteoglycan with a molecular weight of approximately 200 kDa. The protein is translated from 36 exons located on human chromosome 1, p32 and on mouse chromosome 4.^{[29-31]} Agrin may splice between exon 19 and 20 into an amino-terminal half and a carboxy-terminal half The amino-terminal half contains nine domains of Kazal-type protease inhibitor, a single laminin-like region, two cysteine-rich regions, and one serine- and threonine-rich region, while the carboxy-terminal half contains one serine- and threonine-rich region, three identical laminin regions, and four EGF-repeats.^{[32, 33]} Biochemically purified agrin has five main functional fragments with sizes of 150, 135, 95, 70, 50, and 20 kDa.^{[31, 34]} However, many variants of agrin have been reported with inserts of different numbers of peptides at denoted specified locations, “X, Y, and Z” sites, such as Ag_{x=0, 3, 12}/Ag_{y=0, 4, 8, 12}/Ag_{z=0, 8, 11, 19}, indicating that 0, 3, or 12 amino acids can be inserted into the X site, and/or 0, 4, 8, or 12 amino acids can be inserted into the Y site, and/or 0, 8, 11, or 19 amino acids can be inserted into the Z site. Different combinations of these amino acid inserts can generate more than 20 variants of agrin.^{[29, 35, 36]} Ag_{y} segment proteins have been exclusively found in the CNS.^{[37]}

It is well known that agrin induces synaptic formation of the nerve-muscle junction. In cultured neuromuscular tissue, agrin functions as an instructive molecule to order acetylcholine receptor clustering on post-synaptic membranes.^{[38, 39]}

In the central nervous system (CNS), agrin is closely associated with neuronal survival, growth, and synaptogenesis.^{[40-42]} When agrin function is antagonized by antisense oligonucleotides and specific antibody, synaptic formation is severely compromised in hippocampal neurons in culture. Adding recombinant agrin to cultured cells can reverse the antisense and antibody effects.^{[43]} Agrin promotes dendritic elongation and dendritic branching, but limits the elongation rate of main axons without affecting the formation of axonal branches.^{[43]} Interestingly, the amino-terminal half of agrin functions as a stop signal for presynaptic neurons to inhibit neurite outgrowth.^{[44]} Furthermore, deleting the Ag_{z} segment results in smaller brains in homozygous compared to heterozygous mice.^{[45]} Moreover, the formation of glutamatergic and GABAergic synapses is abnormal, and the mice cannot survive after birth.^{[45]} Agrin is a very important molecule in synaptic formation because it communicates with pre- and post-synapses via the extracellular matrix and interacts with other neural growth factors and basal laminal membrane proteins.^{[46-49]} The sequence of an agrin receptor has been reported. (International Patent Application No. WO 95/13298) A change in the level of agrin has also been associated with congenital muscular dystrophy in U.S. Patent Application No. 2003/0024981. A fragment of agrin (about 15 kDa) has been identified as a potential therapeutic agent in controlling seizures due to epilepsy or traumatic brain injury. (U.S. Patent Application No. 2005/0159354).

Agrin may also regulate neuronal responses to excitatory neurotransmitters. One study demonstrated that c-fos expression induced by activation of glutamatergic receptors was significantly lower in agrin-deficient (Ag_{z}-deletion) neurons than in normal neurons from mice.^{[50]} When agrin was given to the agrin-deficient neurons in a very high dose, c-fos expression was still lower than in normal neurons.^{[50]} It was further observed that agrin mRNA expression in a seizure model was affected.^{[51]} These studies suggest that agrin has wider functions in the brain, including synaptic formation, neurite growth, and signal-transduction pathway modulation. One study indicated that agrin gene expression in ciliary ganglion neurons was decreased following postganglionic axotomy in chicks.^{[52]}

NMDA and neuropathic pain. Glutamate activates both metabotropic and ionotropic receptors. Examples of ionotropic receptors include alpha-amino-3 hydro-5-methyl-4-isoxazolepropionic acid (AMPA), kainate, and NMDA receptors.^{[53]} NMDA receptors are further categorized as NR1 and NR2 (NR2A, NR2B, NR2c, NR2D). NMDA receptors play a key role in fetal development and maintenance of physiological function.^{[54]} The activation of NMDA receptors requires glutamate and glycine working together to open NMDA channels. The functional NMDA channel subunits are thought to have an amino-terminal segment and four hydrophobic domains (M1-M4) from two copies of both NR1 and NR2 subunits.^{[55, 56]}

Following tissue or nerve damage, glutamate, aspartate, and SP are dramatically increased in the dorsal horn of spinal cord.^{[57-59]} Glutamate is an NMDA receptor agonist, and SP increases NMDA receptor excitability by removing magnesium blocking^{[60, 61]}, which results in wind-up and hyperexcitability. This observation is supported by the fact that NMDA receptor antagonists, ketamine and MK801, suppress hyperalgesia and allodynia, when given systemically and locally.^{[62, 63]} However, the effects for ketamine and MK801 have been reported by some researchers to only be seen at doses that induce ataxia.^{[64]} However, selective NMDA receptor antagonists yielded lower toxicity. For example, the NMDA2B antagonist, (1S,2S)-1-(4-hydroxy-phynel)-2-(4-hydroxy-4-phenyl-piperidino)-1-propanol, depressed hyperalgesia/allodynia at a dose devoid of adverse effects in animal models of neuropathic pain.^{[65, 66]} NMDA receptors are targets for new drugs for NP modulation.

SCP-1 and SCP-M1, Analgesics for Pain. SCP-1 (2-(1,1-dioxido-3-oxo-1,2-benzisothiazol-2(3H(-yl)-N-(4-hydroxyphenyl)acetamide) is a derivative of acetaminophen formed by chemically linking saccharine and acetaminophen (by reacting sodium saccharide with 2-chloro-N-(4-hydroxyphenyl)acetamide). This novel compound was shown to relieve nociceptive pain without acute liver or kidney toxicity.^{[67]} In studies where SCP-1 was given orally to rodents in doses from 3.7 mmol/kg^{[68]} to 36 mmol/kg (unpublished observation), the animals were still moving normally, while acetaminophen killed all the animals at a dose of 6 mmol/kg. SCP-1 did not induce apoptosis in cultured human hepatocytes at a concentration of 100 μM, as acetaminophen did. Moreover, SCP-1 suppressed formalin- and CFA-induced nociceptive pain in rodent models without any toxicity. When given intrathecally to Bennett model rats with hyperalgesia and allodynia, SCP-1 (30 nmol/kg) also depressed the signs of allodynia and hyperalgesia.^{[69]} SCP-M1 synthesis and structure is described in U.S. Pat. No. 6,806,291. SCP-M1 is an N-acylated-4-hydroxyphenylamine (acetaminophen) derivative resulting from the hydrolysis of SCP-1 to form 2-{[4-hydroxyphenyl)carbamoyl]methylsulfamoyl}benzoic acid. SCP-M1 is reported in U.S. Pat. No. 6,806,291 to possess high analgesic activity in a pain model with transitory inflammatory-induced hyperalgesia.

RNA interference and pain. Double-stranded RNAs have been shown to have an inhibitory effect on the expression of a number of target genes in a variety of organisms. Small, interfering RNA (“siRNA”; usually about 20-25 nucleotides) can be assembled into endoribonuclease-containing complexes known as RNA-induced silencing complexes. The siRNA strands subsequently guide the complexes to complementary RNA molecules (target strands), where the endoribonuclease cleaves and destroys the target RNA strand.^{[70-72]} P2X3 (a subunit of purinoreceptor, a ligand-gated ion channel, activated by ATP) siRNA has been shown in vivo to suppress NP in partial sciatic nerve injury models.^{[73, 74]}

We have shown that the concentration of agrin protein is important in preventing the development of neuropathic pain. The agrin protein and gene expression was shown to be down-regulated in mammals with neuropathic pain. Using well-known rat models for neuropathic pain, increasing either agrin gene expression or agrin protein resulted in suppression of the development of neuropathic pain. Agrin protein can be administered in a number of ways for treating neuropathic pain, including intrathecally administering the 25 kDa, 50 kDa, 70 kDa and 90 kDa fragment of the carboxy-terminal end of agrin. In addition, we have shown that agrin concentration can be modulated by administering certain compounds, e.g., SCP-1, SCP-M1, and MK801. SCP-1 and SCP-M1 were shown to increase agrin concentration. Agrin protein decrease was shown to be blocked by a non-competitive NMDA receptor antagonist (e.g., MK801).

### BRIEF DESCRIPTION OF FIGURES

FIG. 1 illustrates the results of RNA gene-microchip analysis measuring agrin mRNA from lumbar spinal cords dissected from normal rats and from chronic constrictive nerve-injured rats, a neuropathic pain model, at 2 h, 1 day, and 7 days post-injury. (*p<0.05)

FIG. 2A illustrates the results of RT-PCR measuring β-actin (control), and agrin gene expression using primers targeted to the C-terminal of agrin in control (sham-operated, no nerve injury), allodynic, and non-allodynic rats at 3 days and 7 days post injury.

FIG. 2B summarizes the results of measuring the relative intensity of the agrin gene expression bands from FIG. 2A as compared to the expression of β-actin in control, allodynic, and non-allodynic rats at 3 days and 7 days post injury. (*p<0.05; **p<0.01).

FIG. 3A illustrates the results of Western blot analysis measuring β-actin protein (a control) and the agrin protein using antibody to the C-terminal of agrin in control, allodynic, and non-allodynic rats at 3 days and 7 days post injury.

FIG. 3B summarizes the results of measuring the relative intensity of the agrin protein bands from FIG. 3A as compared to the β-actin in control, allodynic, and non-allodynic rats at 3 days and 7 days post injury. (**p<0.01).

FIG. 4A is a set of micrographs of the dorsal horn of rat spinal cord after immunostaining with an agrin antibody which shows the agrin distribution in the dorsal horn from normal (Norm), allodynic (Allo) and non-allodynic (Non-Allo) rats.

FIG. 4B is a set of micrographs of the dorsal horn of rat spinal cord after double immunostaining with an agrin antibody, a growth-associated Protein-43 (Gap43) antibody, and an agrin (Agr510) antibody, a stain for cell nuclei stain (4′,6-diamido-2 phenylindole dihydrochloride, Dapi), and then merging the images to show the relative distribution of these proteins in the dorsal horn from normal rats, with the arrows indicating the co-localization of both agrin and Gap43.

FIG. 4C is a set of micrographs of the dorsal horn of rat spinal cord after double immunostaining with an agrin antibody, a glial fibrillary acid protein (GFAP) antibody, and Dapi, and then merging the images to show the relative distribution of these proteins in the dorsal horn from normal rats.

FIG. 5 (bottom panel) illustrates the results of RT-PCR measuring β-actin (control), and agrin gene expression using primers targeted to the C-terminal of agrin, in rat lumbar spinal tissue cultured with only medium (control), NMDA, or NMDA with Substance P at 6 h and 24 h after treatment, and FIG. 5 (top graph) illustrates the relative intensity of the agrin gene expression bands as compared to the β-actin expression in rat lumbar spinal tissue cultured with only medium (control), NMDA, or NMDA with Substance P for 24 h after treatment. (*p<0.05; **p<0.01).

FIG. 6 (bottom panel) illustrates the results of Western blot analysis measuring β-actin and the agrin protein using antibody to the C-terminal of agrin in rat lumbar spinal tissue cultured with only medium (control), NMDA, NMDA with Substance P, or MK801 (a non-competitive NMDA receptor antagonist, for 6 h and 24 h after treatment; and FIG. 6 (top graph) illustrates the relative intensity of the agrin protein bands using β-actin as a control in rat lumbar spinal tissue cultured with only medium (control), NMDA, or NMDA with Substance P for 6 h and 24 h after treatment. (*p<0.05; **p<0.01).

FIG. 7 illustrates the effect on paw withdrawal threshold for tactile allodynia measured using von Frey filaments for up to 7 days after injury in allodynic rats with injury alone and in allodynic rats injected just prior to injury with SCP-1, APAP, or vehicle (control). (*p<0.05; **p<0.01)

FIG. 8A illustrates the results of RT-PCR analysis measuring β-actin and the agrin gene expression using primers to the C-terminal of agrin in lumbar spinal tissue from normal rats, and in allodynic rats injected just prior to injury with SCP-1 or vehicle (control), and sacrificed for mRNA at 1, 3 and 7 days post-injury.

FIG. 8B illustrates the relative intensity of the agrin gene bands from FIG. 8A as compared to the β-actin band in normal rats and in allodynic rats injected just prior to injury with SCP-1 or vehicle (control), and sacrificed for mRNA at 1, 3 and 7 days post-injury. (*p<0.05; **p<0.01).

FIG. 9A illustrates the results of Western blot analysis measuring β-actin and the agrin protein using antibody to the C-terminal of agrin in lumbar spinal tissue from normal rats, and in allodynic rats injected just prior to injury with SCP-1, APAP, or vehicle (control), and sacrificed for mRNA at 1, 3 and 7 days post-injury.

FIG. 9B illustrates the relative intensity of the agrin protein bands from FIG. 9A as compared to β-actin in normal rats and in allodynic rats injected just prior to injury with SCP-1 or vehicle (control), and sacrificed for mRNA at 1, 3 and 7 days post-injury. (*p<0.05; **p<0.01).

FIG. 10A is a set of micrographs of the dorsal horn of rat spinal cord after immunostaining with an agrin antibody which shows the agrin distribution in the dorsal horn from normal, non-allodynic (injured but no allodynia) and allodynic rats injected just prior to injury with SCP-1, APAP or vehicle (control), and sacrificed 7 days post-injury.

FIG. 10B is an enlarged version of each of the left dorsal horns shown in FIG. 10A.

FIG. 11 illustrates the paw withdrawal threshold for tactile allodynia measured using von Frey filaments for up to two hours in allodynic rats injected on day 7 after injury (time 0) with several concentrations of SCP-1, ranging from 1 nmol to 200 nmol.

FIG. 12 illustrates the paw withdrawal threshold for tactile allodynia measured using von Frey filaments for up to eight hours in allodynic rats injected on day 7 after injury (time 0) with SCP-1, APAP, SCP-M1 (JMM), or vehicle (control).

FIG. 13 illustrates the effect on thermal hyperalgesia, measured as percentage increase in paw withdrawal latency, for up to two hours in allodynic rats injected on day 7 after injury (time 0) with SCP-1, APAP, or vehicle (control).

FIG. 14A (top left panel) illustrates the results of RT-PCR measuring agrin gene expression using primers targeted to the C-terminal of agrin in rat lumbar spinal tissue from normal rats, and allodynic rats injected at 7 days post nerve injury with SCP-1 or vehicle (control), and sacrificed at 1 hr and 2 hr post-injection; and FIG. 14A (bottom left panel) illustrates the relative intensity of the agrin gene expression bands as compared to β-actin in the RT-PCR. (**p<0.01).

FIG. 14B is a micrograph of the dorsal horn from an allodynic rat spinal cord with immunostaining for agrin after 1.5 hr SCP-1 after intrathecal injection at 7 days post nerve injury.

FIG. 15A is a set of micrographs of the dorsal horn of rat spinal cord after immunostaining with an agrin antibody which shows the agrin distribution in the dorsal horn from normal, non-allodynic (injured but no allodynia) and allodynic rats injected at 7 days post-injury with SCP-1, SCP-M1 (JMM), APAP, and vehicle, and sacrificed 2 h after injection.

FIG. 15B is an enlarged version of each of the left dorsal horns shown in FIG. 15A.

FIG. 16 illustrates the paw withdrawal threshold for tactile allodynia measured using von Frey filaments for up to 4 h in allodynic rats injected at 7 days after injury (time 0) with SCP-1, vehicle (control), SCP-1 plus agrin siRNA, or SCP-1 plus agrin nFsiRNA.

FIG. 17A (top panel) illustrates the results of Western blot analysis (top panel) measuring β-actin and the agrin protein using antibody to the C-terminal of agrin in lumbar spinal tissue from normal rats, and in allodynic rats injected at 7 days after injury with SCP-1, vehicle (control), SCP-1 plus agrin siRNA, and sacrificed at 2 or 4 h post-injection; and FIG. 17A (bottom graph) illustrates the relative intensity of the agrin protein bands compared to β-actin in the Western blot results. (**p<0.01).

FIG. 17B (top panel) illustrates the results of Western blot analysis (top panel) measuring β-actin and the agrin protein using antibody to C-terminal of agrin in lumbar spinal tissue from normal rats, and in allodynic rats injected at 7 days after injury with SCP-1, vehicle (control), SCP-1 plus agrin nFsiRNA, and sacrificed at 2 h post-injection; and FIG. 17B (bottom graph) illustrates the relative intensity of the agrin protein bands as compared to β-actin in the Western blot results. (**p<0.01).

FIG. 18A is a set of micrographs of the dorsal horn of rat spinal cord after immunostaining with a p-NR1 antibody which shows the number of p-NR1 labeled neurons in the ipsilateral dorsal horn from normal rats and allodynic rats injected with SCP-1, SCP-M1 (JMM), vehicle, SCP-1 plus siRNA, and APAP, and sacrificed 2 hr after injection.

FIG. 18B is an enlargement of the dorsal horn area shown in the corresponding FIG. 18A that indicates the p-NR1 neurons.

FIG. 19 illustrates the average number of p-NR1 labeled neurons in the ipsilateral dorsal horn from normal rats and allodynic rats injected seven days after injury with SCP-1, SCP-M1 (JMM), vehicle, SCP-1 plus siRNA, and APAP, and sacrificed 2 hr after injection. (*p<0.05; **p<0.01)

### EXAMPLE 1

### Materials and Methods

Animal model: All animals were maintained under the supervision of licensed laboratory veterinarians and experienced animal-care technicians. All rats were purchased from Charles River Laboratories (Wilmington, Mass.). Surgical procedures were performed under anesthesia by 1.5% isoflurane with a mixture of 50% oxygen and 50% nitrous oxide delivered by means of an open-mask system at 1.5 l/minute. Body temperature was maintained during the procedures at 38±0.5° C. by an automatic heating device.

Rat neuropathic pain models generated by partial sciatic nerve injury displayed allodynia (a painful reaction to innocuous stimuli such as touch, warmth, slight pressure), hyperalgesia, paw shaking, and paw licking after sciatic nerve injury. Such abnormal symptoms somewhat mimic those of patients with neuropathic pain seen in clinical studies. Therefore, these rat models are widely used for neuropathic pain research. The allodynia could last 1-3 weeks.^{[75]}

Bennett Model: In this model, approximately 0.8 cm of the left sciatic nerve was exposed and freed with sharpened scissors at middle-thigh level as described by Bennett and Xie.^{[76]} Four ligatures were placed loosely around the sciatic nerve with 4-0 chromic catgut, with 1 mm space between the ties, which compresses the nerve but epineural blood circulation is preserved. The tissue and skin were closed in layers. After the nerve injury, most animals displayed allodynia and hyperalgesia from day 5 to day 60. Most allodynia was seen during the time period of 7-14 days after the surgery.

Gazelius model: The incidence of allodynia/hyperalgesia for the Bennett model was usually 30-50% of all nerve-injured animals; however, sometimes the incidence dropped to 10% or lower for unknown reasons. Therefore, another neuropathic pain model where the sciatic nerve is photochemically injured, (the Gazelius model^{[77]}) was used. This model displayed higher magnitude and longer duration of allodynia and hyperalgesia than other models, with an allodynic incidence higher than 90%. Erythrosin B, used for inducing photochemical injury to the sciatic nerve, is a light-sensitive dye that, in a blood vessel, can react to a laser beam to produce single oxygen molecules in the endothelial surface, resulting in intense platelet aggregation, micro-emboli, and infarction.^{[78]}

The left sciatic nerve was exposed at middle-thigh level corresponding to the Bennett model, and then a thin aluminum foil was placed under the nerve to reflect the laser light. A low-energy, green laser beam (4.5 mW output on the tip of the transmitter fiber) with a wave-length of 532 nm, was focused on the sciatic nerve about 0.5 mm above. The nerve was irradiated for 20 min immediately following an intravenous erythrosin-B injection (32.5 mg/kg, Sigma, St. Louis, Mo.). After the irradiation the sciatic nerve appeared pale, and the blood in the vessels of the nerve segment was coagulated. The tissue and skin were then closed in layers.

Intrathecal catheter installation: A PE10 catheter, stretched to a somewhat thinner diameter in the distal part, was introduced into the spinal canal (subarachnoid space) via a 21 G (gauge) cannula between the L5 and L6 lamina and advanced to the lumbar enlargement in rats. The proximal part was tunneled under the skin and led out on the upper dorsal region. The location of the catheter was verified by the injection of 6 μl (50 mg/ml) xylocalne, such that a transient, flaccid paralysis of the legs was produced, and by X-ray after administration of 10 μl iodine contrast medium (Astra, Sweden). The catheter could be kept for up to two weeks in animals.^{[79]}

Intrathecal injection of SCP-1 and SCP-M1: Although SCP-1 and SCP-M1 do not dissolve readily in water, 45% 2-hydroxypropyl-α-cyclodextrin in saline can dissolve them. For example, SCP-1 (3.32 mg), SCP-M1 (JMM) (3.5 mg), or acetaminophen (1.51 mg) was dissolved in 1 ml of saline with 45% cyclodextrin to provide a final concentration of 10 mM. Either SCP-1, SCP-M1 (JMM) or acetaminophen (at various concentrations in 10 μl) were injected via the PE10 catheter. The maximum volume injected at any one time was 10 μl.^{[79]} The solubility of SCP-M1 is very similar to that of SCP-1, and the same concentrations were used as stated above for SCP-1.

Behavioral study: Animals were allowed to adapt to the experimental environment (a quiet room with daylight-like illumination) three times before collecting data. All of the data collections were performed between 9:00 AM and 4:00 PM to minimize time cycles and other behavioral interference. In each individual experiment, the animal was allowed to adapt for 15 min before the test started.

Tactile allodynia test: In a transparent glass chamber (20×20 cm) with a mesh net floor, each animal was subjected to the tactile allodynia test. A positive response is when an innocuous tactile stimulus induces a brief paw withdrawal away from the stimulation, similar to a paw withdrawal due to heat. A set of von Frey filaments from 0.01 to 28 g (Stoelting Co., Wood Dale, Ill.) were applied to the mid-paw of the allodynic rats at 1 gram, and then progressing up or down. The filament was pressed against the plantar surface for 1 to 2 seconds until it bent. Subsequent applications were made with a higher or lower von Frey filament depending on the animal response. The testing always began with normal paws. If a filament induced a brief paw withdrawal of a paw without bending in three out of five tests, the size of the filament was recorded as eliciting a positive withdrawal threshold.^{[80]} Allodynia was considered to be present when a withdrawal threshold was evoked by a filament corresponding to 8 grams or less.

Thermal hyperalgesia test: The thermal hyperalgesia test device was composed of a chamber (20×20 cm) with a glass bottom, whose temperature was adjustable via a wire and a light beam for focusing on a paw plantar surface (Plantar Analgesia Meter, IITC LifeSci, Woodland Hills, Calif.). When a button was pushed, the intensity of the light beam was increased to induce paw withdrawal. The temperature of the glass bottom was set at 28° C., and the intensity of the light beam was set at 35% of the light source. The resulting light beam caused a temperature of about 42° C. on the plantar of the rat when the cutoff time was set for 12 sec. The latency time in seconds from the onset of the intense light beam to paw withdrawal was defined as the withdrawal threshold of the paw. Two consecutive tests were averaged to establish the threshold. The values obtained in uninjured paws were compared with those in nerve-ligated paws. If a latency value of an injured paw was 30% lower than that of an uninjured paw, the animal was considered to be thermally allodynic and/or hyperalgesic.

Gene expression alterations after the nerve injury: Gene expression for amino(“N”)- and carboxy(“C”)-terminal halves of agrin was analyzed from L3-5 spinal cord of the rats (sacrificed at 2 hr, 24 hr, 3 days, and 7 days post surgery), using RT-PCR and real-time PCR. The differences in gene expression between sham-operated and nerve-injured groups were compared.

Total RNA extraction: The rats were deeply anesthetized with isoflurane and decapitated. The spinal cord from L3-5 was quickly removed and immediately put into a tube containing TRIzol® (2 ml, Invitrogen, Carlsbad, Calif.). The tissue was homogenized with IKA® T8 homogenizer (IKA®, Staufen, Germany) for 2 min, allowed to stand at room temperature for 3 min, and 0.4 ml chloroform added. The tube was vortexed and centrifuged at 12,000×g for 15 min. The water phase was transferred to a clean tube without any contamination from other phases. Then 1 ml isopropanol was added to the water phase, and the mixture allowed to stand for 10 min at room temperature. The tube was centrifuged for 10 min at 12,000×g. The supernatant was discarded, and the pellet washed with 1 ml 70% ethanol. The ethanol was discarded, and the RNA was re-dissolved in 30 μl nucleotide-free water. A 1 μl aliquot was taken and reserved to dilute 20-fold for RNA quality control. The remainder was stored at −80° C. A biophotometer (Eppendorf, Germany) and RNA gene chip analyzer (2100 Bioanalyzer, Agilent, Waldbronn, Germany) was used to measure the RNA concentration and quality. Only RNA with A_{260/280}>2.0 and/or a Ratio_{18S/28S}>1.4 was used for the experiments.

One-step RT-PCR. A total volume of 50 μl of the above RNA solution was used for the reaction with reverse-transcriptase polymerase with 1 μg RNA, 25 μl buffer, 1 μl RT/Platinum Taq Mix (SUPERSCRIPT™, Invitrogen), 2 μl MgSO₄ (50 mM) and 2 μl primers (10 μM). The reaction was run for 22 min at 42° C., 2 min at 95° C., followed by 36 cycles of 95° C. for 30 sec, 60° C. for 30 sec and 72° C. for 1 min. All primers were synthesized by Invitrogen (Carlsbad, Calif.). The forward primer for the N-terminal half of agrin was 5′-TGGCAGTGACGGTGTTGACTAC-3′ (SEQ ID NO: 1) and the reverse primer was 5′-CACGGCGGGACAGGCATAC-3′ (SEQ ID NO:2) (741-1144 nt). The forward primer for the C-terminal half was 5′-TCAGGAGCAAAGAGCCCATAGC-3′(SEQ ID NO:3), and the reverse was 5′-ATGTAGGTCCGCCCATCAAAGG-3′(SEQ ID NO:4) (5012-5510 nt).

Real-time PCR (Bio-Rad) quantified the N- and C-terminal halves of agrin with a set of different primers (Forward and Reverse for N-half: 5′-AACCAGTCTCGCAACATCAATCTTC-3′ (SEQ ID NO:5); 5′-GCCTCCTCATCTCCACTCAGTTC-3′ (SEQ ID NO:6), 3027-3110 nt, and Forward and Reverse for C-half: 5′-CCCACCCTCCGAGCCTACC-3′ (SEQ ID NO:7); 5′-GCCCATCCAACAGAGCCAGAG-3′ (SEQ ID NO:8), 4030-4159-nt.

Total RNA (5 μg) from each sample was synthesized for cDNA with first-strand cDNA synthesis kit (Amsham). cDNA (1 μl) was serially diluted 4 fold. Each dilution was loaded into triple wells for 45 cycles with a SYBER-GREEN™ (Bio-Rad). The results were analyzed by Real-Time PCR software (Bio-Rad, Hercules, Calif.).

Probe synthesis and Gene-chip hybridization: RNA (8.5 μg) from each sample (3 rats for each group) was used for first- and second-strand cDNA synthesis with Double-strand cDNA kit (Invitrogen). The double cDNAs were converted to cRNAs using Enzo BIOARRAY-HIGHYIELD™ RNA Transcript Labeling Kit. The cRNAs were biotinylated and segmented into about 200- to 400-nt probes. The probes were hybridized against U34A gene-chip DNA arrays. Expressed sequence tag clusters were analyzed by UniGene database Build 74 (Affymetrix, Santa Cruz, Calif.).

Gene-expression alterations after intrathecal drugs and nerve injury: All the procedures were the same as the above except for a 10 μl intrathecal injection of either SCP-1, SCP-M1 (JMM), acetaminophen, or saline at the desired concentration immediately prior to nerve injury. Changes in agrin gene induced by the injected drug was analyzed and compared.

Spinal cord tissue culture: The dorsal horn of fresh rat lumbar spinal cord was cut into 1-mm sections with a sharp blade under sterile conditions and maintained in culture in 12-well plates with DMEM and 10% FBS. After 2 days, 100 μM NMDA was added to 6 wells. The other 6 wells were used as controls. The culture was maintained for 24 hr, and the tissue subjected to RNA and protein extraction as well as RT-PCR and Western blotting to examine alterations of agrin gene and protein, as described in this Example.

Western blot for detecting protein expression of agrin: Agrin protein was analyzed from L3-5 spinal cord with Western blot as described below at times of 1 day, 3 days, and 7 days post-surgery. The changes in these proteins between sham-operated, nerve-injured (no drug), and nerve-injured (pre-emptive drug) groups were analyzed.

Agrin Protein Extraction: The rats were deeply anesthetized with isoflurane and decapitated. Spinal cord L3-5 was quickly removed and immediately placed into tissue lysis buffer (1 ml, Sigma Co., St. Louis, Mo.) that contained protein enzyme inhibitors (1:100, Halt, Pierce). The tissue was homogenized for 2 min and incubated on ice for 45 min. The sample was then centrifuged at 10,000×g for 30 min at 4° C. The supernatant was transferred to a new tube and centrifuged again for further clarifying. The concentration of the protein was determined by an Agilent's Bio-Rad protein chip (Agilent's 2100 bioanalyzer, Santa Clara, Calif.) and by the Bedford method (reagents from Bio-Rad).

Western blot: Protein (20 μg) from each sample was mixed with 2× electrophoresis buffer and boiled for 5 min. The samples, along with a negative control, were loaded on 4-15% acrylamide tris-glycine SDS gels and electrophoresed in a cell (Bio-Rad) for approximately 1 hr. A Hybond-P polyvinylidene difluoride membrane (Bio-Rad, Hercules, Calif.) was used for transferring the protein in a mini-trans-blot transfer cell (Bio-Rad) for 2 hr. The membrane was then soaked in 2% milk for 1 hour and probed with monoclonal agrin antibody (Agr510 and Agr530, 1:5000, StressGen Biotechnologies Corp., Victoria, Canada) or β-actin antibody (1:5000) at 4° C. overnight with continual shaking. The following day goat anti-mouse secondary antibody conjugated to Alexa Fluor680 (1:5000, Molecular Probe) was added to the membrane and incubated for 1 hr at room temperature. The samples were washed three times with TBS buffer (Tris Buffered Saline, DAKO, Carpinteria, Calif.) between steps. The membrane was then scanned with an Odyssey (LI-COR) scanner and analyzed by Odyssey software (Li-Cor, Lincoln, Nebr.).

Immunohistochemistry: At 7 days post-surgery, the animals were deeply anesthetized with isoflurane and perfused via left ventricle with 100 ml warmed saline, followed by perfusion with 50 ml warmed 4% paraformaldehyde (pH 7.4) and then 250 ml cold 4% paraformaldehyde. The L4-5 spinal cords from rats sham-operated, nerve-injured, or nerve-injured with preemptive intrathecal treatment with either 30 nmol/kg SCP-1 in 10 μl saline, 30 nmol/kg acetaminophen at 10 μl volume, or 10 μl saline, and allodynic rats treated with SCP-1, SCP-M1 or SCP-1 plus agrin siRNA were dissected out and cut into 20 μm sections with a cryostat (Shandon, USA). The sections were mounted on a slide and stored at −80° C. To process for staining, the sections were air-dried for 45 min, and then washed three times with PBS for 10 min. The sections were then blocked with 10% serum (compatible with the species-specific secondary antibody) for 1 hr, with 3 washes in between. Primary antibody against either agrin (Agr510 and Agr530, 1:8000, StressGen Biotechnologies Corp., Victoria, Canada), GAP43 (1:1000, Santa Cruz Biotechnology, Inc., Santa Cruz, Calif.), or GFAP (1:1500, AbD Serotec, Raleigh, N.C.) was incubated with the sections overnight at 4° C. After 3 washes, secondary antibody conjugated with FITC or Cy3 was applied to visualize the stain. Then the sections were washed thoroughly and mounted under cover-glass for microscopy. Some sections were subjected to double staining (agrin+GFAP, or agrin+GAP43). An Axioplan2 fluorescence microscope (ZEISS®, Germany) with deconvolution function was used for histological image.

Agrin RNA interference development and intrathecal injection: Agrin siRNA was designed and produced according to the Ambion protocol (Ambion, Austin, Tex.). Beginning with the AUG start codon of Agrin mRNA, a search was conducted for AA dinucleotide sequences. Each AA and the 3′ adjacent 19 nucleotides were recorded as siRNA target sites. To obtain more antagonism of the agrin gene, the siRNA chosen contained about 30-50% GC, less than 4 A's or T's (which act as a termination signal for RNA polymerase III), and 2-4 targets on the mRNA. In addition, a negative control siRNA with the same nucleotide composition but different from the order of the C-terminal agrin mRNA (nFsiRNA) was used for the experiments:

For N-terminal agrin: Position in gene sequence 858; GC content 48.1%.

(SEQ ID NO: 9)

| | |
|---|---|
| Sense strand RNAi: | CCUAGUGUUGAGGAUCCAGtt |

(SEQ ID NO: 10)

| | |
|---|---|
| Antisense strand RNAi: | CUGGAUCCUCAACACUAGGtt |

For C-terminal agrin: Position in gene sequence 5994; GC content 48.1%.

(SEQ ID NO: 11)

| | |
|---|---|
| Sense strand RNAi: | GCCCUCAAAGUCCUGUGAUtt |

(SEQ ID NO: 12)

| | |
|---|---|
| Antisense strand RNAi: | AUCACAGGACUUUGAGGGCtt |

In rats with tactile allodynia (a withdrawal threshold with a filament below 8 g), an intrathecal catheter (PE 10) was installed and linked to an osmotic pump (Alzet) in a 37° C. water bath. Following a bolus injection of SCP-1 (30 nmol/kg, 10 μl), agrin siRNA (100 nM) was intrathecally administered continuously by the pump at a rate of 5 μl/1 hour for 4 hr. The animals were subjected to von Frey filament test every 15 min for 4 hr to determine whether the effects of SCP-1 on allodynia suppression are blocked by agrin siRNA. A control siRNA (nFsiRNA) was applied in the same way. The behavioral data was analyzed for agrin effects. Immediately after behavioral experiments, the animals were sacrificed and subjected to protein extraction of lumbar spinal cord, followed by Western-blot analysis as described above.

### EXAMPLE 2

### Down-Regulation of Agrin Following Sciatic Nerve Injury

The L3-5 spinal cords were removed quickly from rats sacrificed at 1, 3, and 7 days post chronic constrictive nerve injury (“CCI,” Bennett model) and subjected to isolation of total RNA using TRIzol reagent, using methods as described in Example 1. RNA concentration and quality were controlled by Eppendorf spectral photometer (A_{260/280}≧2.0; Eppendorf, Hamburg, Germany), and Agilent's 2100 bioanalyzer (RATIO_{18S/28S}>1.4). Only the RNA samples meeting high quality criteria were used in the experiments.

Rat agrin mRNA is 7286 bp, which encodes an approximately 200-kDa protein. Although the agrin mRNA can generate many variants by splicing at different sites, there are five main functional agrin proteins: Ag150, Ag135, Ag95, Ag70, Ag50 and Ag20. In a first step, gene expression was analyzed in the N-terminal half and in the C-terminal half, corresponding to a sequence from 216 to 3633, and from 3634 to 6024, respectively.

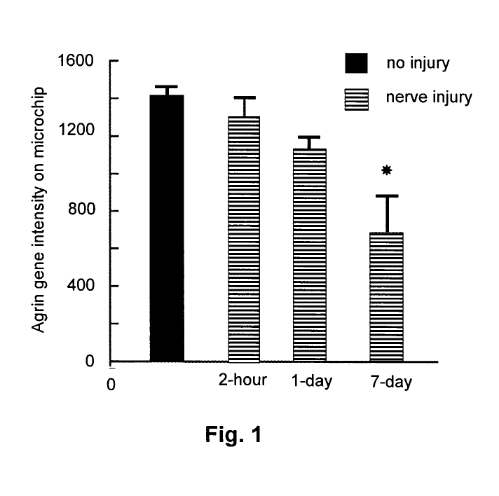

RNA gene-chip (U34A, Affymetrix, Inc., Santa Clara, Calif.) analysis showed the decrease in agrin mRNA from nerve-injured lumbar spinal cords at 2 h, 1 day, and 7 days post-injury. In FIG. 1, agrin gene expression was measured from total RNA from L3-5 spinal cords by microchip (n=3, for each group) from normal (no injury) and chronic constrictive nerve-injured (CCI) rats at time periods of 2 h, 1 day, and 7 days post injury. Total RNA was converted to cDNA with Superscript double strand cDNA kit (Invitrogen kit). The double cDNA was converted to cRNA using Enzo BIOARRAY-HIGHYIELD® RNA transcript labeling kit, and then biotinylated and segmented into probes that were hybridized against U34A gene-chip DNA arrays, and analyzed as described above in Example 1 (Probe synthesis and Gene-chip hybridization). As shown in FIG. 1, agrin mRNA was down-regulated 50% in nerve-injured rats at 7 days, as compared to normal rats. The difference between normal rats and CCI rats at 7 days was significant (p<0.05, Mann-Whitney Test).

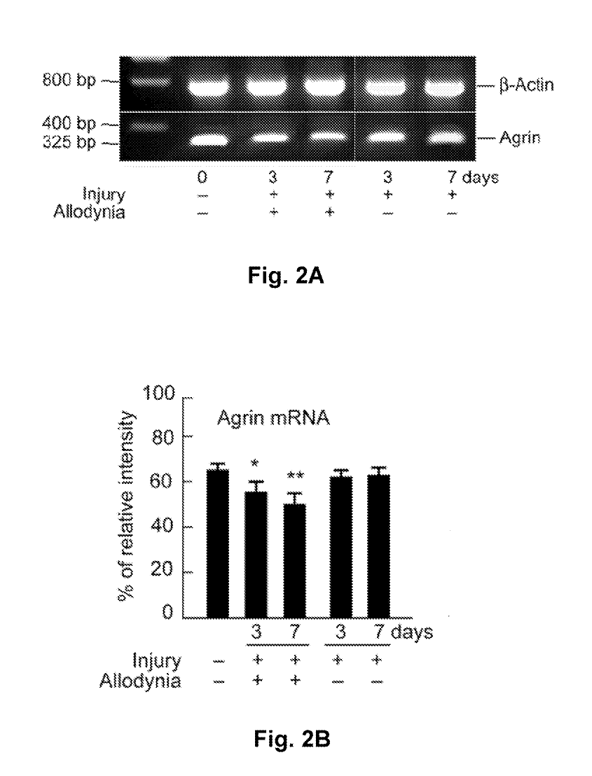

The RNA samples were subjected to RT-PCR using the two sets of specific agrin primers which target gene sequence 741-1144 of the N-terminal half and 5012-5510 of the C-terminal half as described above in Example 1. The results are shown in FIGS. 2A and 2B. In FIG. 2A, the upper panel illustrates the β-actin primers as the PCR control, and the lower panel is the agrin primer for the C-terminal (5012-5510 nt). The C-terminal agrin gene was visually down-regulated only in allodynic rats at 3 and 7 days post-injury, while no agrin gene change was observed in non-allodynic rats. The N-terminal agrin gene had no obvious change after nerve injury. (Data not shown). The control, β-actin, did not change. As shown in FIG. 2A, in allodynic rats, the decrease in agrin gene expression started at 3 days post-injury, with the lowest level occurring at 7 days (the longest time at which the test was performed).

FIG. 2B illustrates the relative intensity of the agrin mRNA bands (C-terminal agrin primers) compared to that of the β-actin band, a house-keeping gene. The Agrin mRNA band relative intensity decreased significantly in the allodynic rats at 3- and 7-day post-injury over the non-allodynic rats and the no injury rats (n=10, *p<0.05 and **p<0.01, respectively, using a two-way Student's t test).

The agrin gene-expression down-regulation was consistent with the time course of allodynia development that started to appear at 3-5 days and that reached a maximal magnitude at 7-14 days post-CCI. These results indicate that regulation of the expression of agrin gene plays a crucial role in NP development.

### EXAMPLE 3

### Agrin Protein Changes Following Nerve Injury

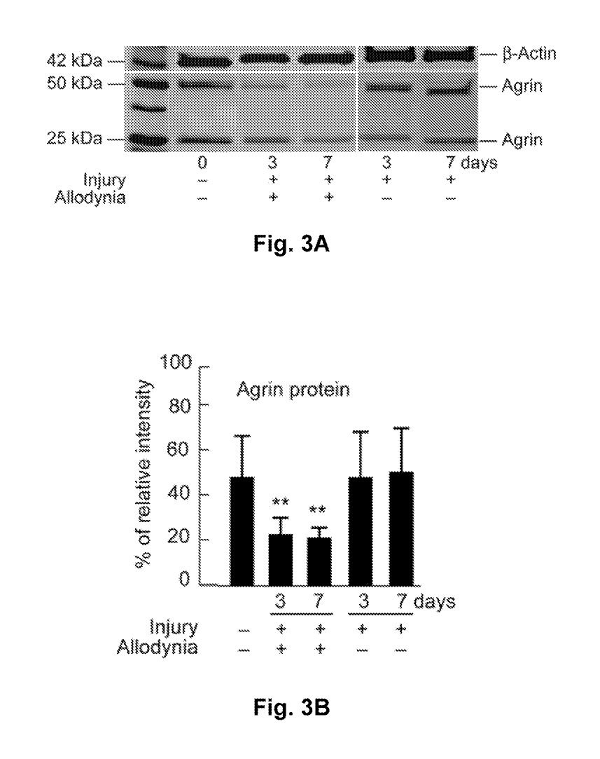

Following the left sciatic nerve injury as in Example 2, agrin protein in the L3-5 spinal cords was found to decrease in allodynic rats as shown by Western blot. FIG. 3A shows that agrin protein decreased in the spinal cord (L3-5) following chronic constrictive injury to the left sciatic nerve. Carboxy-terminal agrin protein fragments of 25 kDa and 50 kDa decreased at days 3 and 7 post-injury in allodynic rats, compared to that of sham-operated, no injury rats and non-alloydynic rats. (FIG. 3A) In FIG. 3A, the β-actin protein (42-kDa band) is shown as a control. In FIG. 3B, the agrin protein is expressed as a percent of relative intensity of the β-actin protein for 8 samples. Because allodynia cannot be discerned before day 3 post-injury, data were collected on or after day 3 post-injury. As shown in FIG. 3B, the agrin decrease in allodynic rats was significant at 3 and 7 days post injury in comparison with non-allodynic rats and no injury rats (**p<0.01, using a two-way Student's t test).

Agrin protein decrease is the corollary of agrin gene down-regulation following nerve injury as shown in Example 2. Agrin antibodies AGR510 and AGR530 (StressGen) were used in the Western blot analysis. According to the antibody manufacturer, the epitope of AGR510 is located approximately at the second laminin region of the C-terminal half, and the epitope of AGR530 is approximately at the first EGF region of the C-terminal half. Thus both label the C-terminal half of agrin. Surprisingly, the two antibodies revealed the same pattern of agrin down-regulation in allodynic rats. Not only did the 25 kDa agrin decrease, but also the 50 kDa agrin, previously reported in the CNS^{[81]}, was down-regulated from day 3 post injury. One sample even showed a decrease at day 1. These results suggest that the segments of agrin corresponding to 25 kDa and 50 kDa are the main functional molecules in pain modulation. It is unlikely that the 50 kDa segment is the dimer of the 25 kDa moiety because the experiments were repeated four times with high concentrations of beta-mercaptoethanol and SDS in the sample buffer to reduce disulfide bonds, and each time the results were the same.

In nerve-injured, non-allodynic rats, no obvious agrin down-regulation was observed at 3 and 7 days post-injury. FIGS. 3A and 3B also show the agrin protein from non-allodynic rats. Neither the 25- nor 50 kDa agrin proteins decreased in the spinal cord at 3 and 7 days post-injury, when compared to normal rats. β-Actin protein (42 kDa band) was used as a control.

These results indicate that when agrin does not decrease, then NP does not occur. This further supports that agrin is a crucial molecule in NP.

### EXAMPLE 4

### Agrin Distribution in Spinal Cord

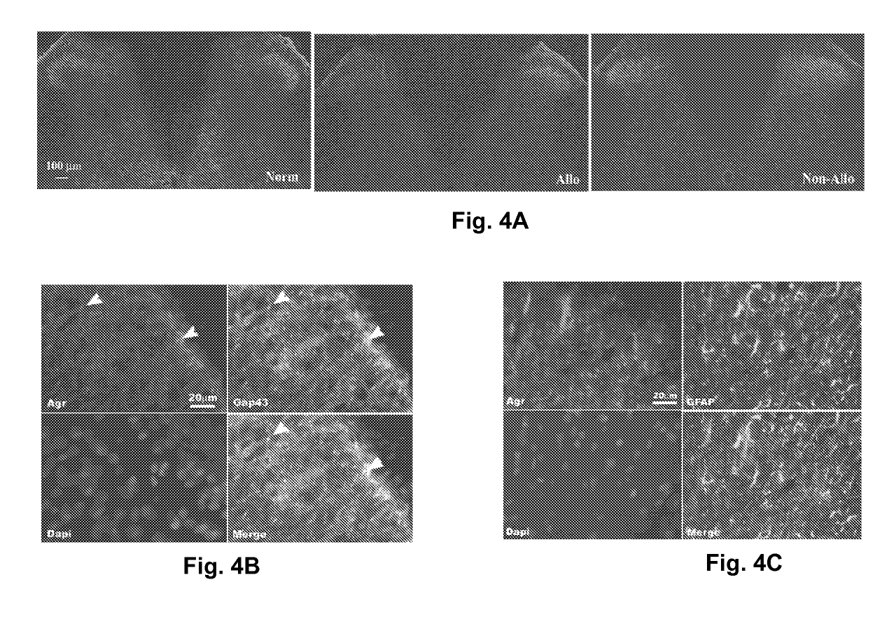

Immunostaining was used to visualize the location and amount of agrin in the spinal cord. Rats (normal, allodynic, and non-allodynic) were perfused with 4% paraformaldehyde at 7-day post-injury, and dissected for the L4-5 spinal cord (n=4, for each group). These spinal cord samples were cut at 20 μm with a cryostat, and stained with agrin antibody as described in Example 1. FIG. 4A shows the results as seen in the dorsal horns. In normal rats, agrin immunoreactivity was bilaterally symmetrical in the dorsal horn, most concentrated on lamina I-III. (FIG. 4A, top panel “Norm”). In allodynic rats, agrin immunoreactivity appeared much lower overall than in normal rats and was particularly low in lamina I-III of the dorsal horn ipsilateral to the injury than in that of the dorsal horn contralateral to the injury. (FIG. 4A, compare Norm to Allo). The lucent space between Lamina I and II was larger in the ipsilateral than in the contralateral dorsal horn in the allodynic rats than in the normal rats. (FIG. 4A) The non-allodynic rats did not show a decrease in agrin immunoreactivity, but were similar to normal rats. (FIG. 4A, compare Norm to Non-Allo).

Growth-associated Protein-43 (Gap43) antibody is a marker for pre-synaptic membranes of neurons. When Gap43 and agrin antibody were used in double staining, agrin and Gap43 were co-localized in the dorsal horn, including lamina I, II, III, and IV. (FIG. 4B, arrows indicating colocalization of agrin and Gap43) The tissues were also stained with Dapi (4′,6-diamido-2 phenylindole dihydrochloride), a compound that binds to DNA and is used to stain cell nuclei. These results indicate that agrin is located on the pre-synaptic membranes of neurons of the dorsal horn. In contrast, GFAP (glial fibrillary acidic protein) antibody is a marker for the non-neural glial cells. When GFAP and agrin antibodies were applied, GFAP did not co-localize with agrin, indicating that agrin is not located in glial cells (FIG. 4C). These data again support that agrin is localized on neurons.

### EXAMPLE 5

### Decrease in Agrin Gene Expression is Induced by NMDA Receptor Activation

Following peripheral nerve injury, large amounts of glutamate, aspartate, and substance P are released into the extracellular space and excite post-synaptic neurons. This results in a long-term hyperexcitability in the spinal center, which is a main cause of NP. Exogenous NMDA and substance P (“SP”) were used to culture rat lumbar spinal tissue. The normal dorsal part of lumbar spinal cord was cut into 0.5 mm slices and cultured in DMEM plus 10% FBS serum. After culturing for 24 h, the slices were treated with 100 μM NMDA with or without 10 nM substance P. Control slices were not treated with NMDA or substance P. Each group (experimental and control) had six samples. At 6 h and 24 h after treatment, the slices were subjected to RNA and protein extraction. The extractions were then assayed for agrin mRNA and agrin protein as described above in Examples 1-3. Briefly, the agrin mRNA was assayed using RT-PCR using C-terminal agrin primers for 36 cycles; and the agrin protein was assayed using Western Blot.

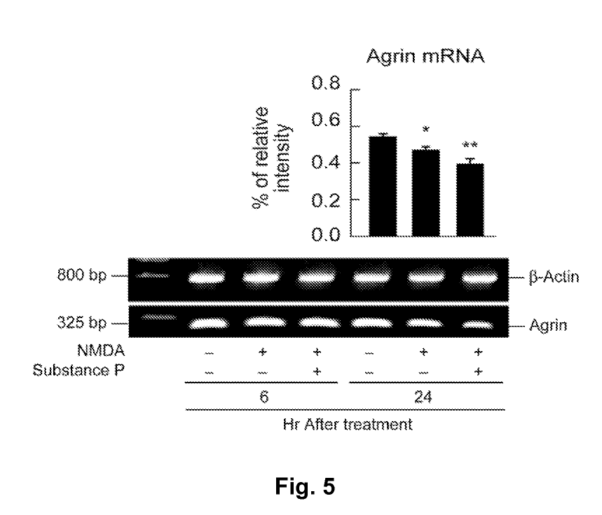

The results of the RT-PCR are shown in FIG. 5 (bottom panel), using β-actin as a control (800 bp). At 24 h, agrin gene expression was visually less in the NMDA-treated samples, when compared to controls without NMDA. SP addition enhanced the down-regulation by NMDA, suggesting that the agrin gene is affected by intense excitation of NMDA receptors. No decrease in agrin mRNA was observed in the 6 h samples in either NMDA group (with or without SP).

FIG. 5 (top graph) illustrates the relative intensity of the agrin mRNA band as compared to β-actin bands at 24 h post-treatment. The mRNA relative intensity was significantly less than the control in both the NMDA slices and in the NMDA/SP slices (*p<0.05 and **p<0.01, respectively, using two way Student's t test).

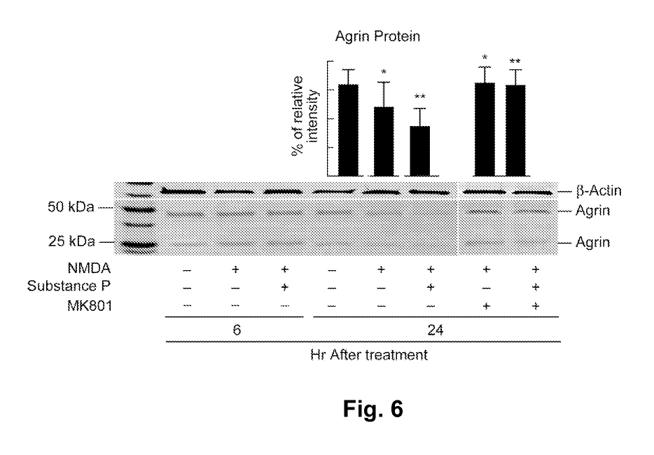

FIG. 6 shows the results of the Western blot analyses for agrin protein. The pattern of agrin protein was consistent with the agrin mRNA discussed above. The protein bands show a decrease in agrin in the 24 h samples in both the NMDA and NMDA/SP samples (FIG. 6, top panel). FIG. 6 (top graph) illustrates the relative intensity of the agrin protein band as compared to β-actin bands at 24 h post-treatment. The agrin relative intensity was significantly less than the control in both the NMDA slices and in the NMDA/SP slices (*p<0.05 (n=6) and **p<0.01 (n=6), respectively, using two way Student's t test). In addition, when 20 μM MK801 (a non-competitive NMDA receptor antagonist which reduces NMDA receptor excitability by inhibiting calcium influx) was added in the culture with either NMDA alone or NMDA plus substance P, it blocked agrin decrease resulting from NMDA receptor over-stimulation. The relative intensity of agrin protein bands to β-Actin bands at 24 hr post treatment showed a significant difference between NMDA and MK801 plus NMDA (n=6; p<0.05), and between NMDA plus SP and MK801 plus NMDA and SP (n=6, p<0.01; two ways Student's t test).

These results demonstrate that in vitro agrin down-regulation is mediated at least in part by an increase in NMDA receptor activation, an effect that is intensified with addition of substance P. It also shows that addition of an NMDA receptor antagonist can prevent some down-regulation of the agrin.

### EXAMPLE 6

### Pre-Emptive Intrathecal Drug Injection Affects Development of Neuropathic Pain and Up-Regulates Expression of the Agrin Gene and Proteins

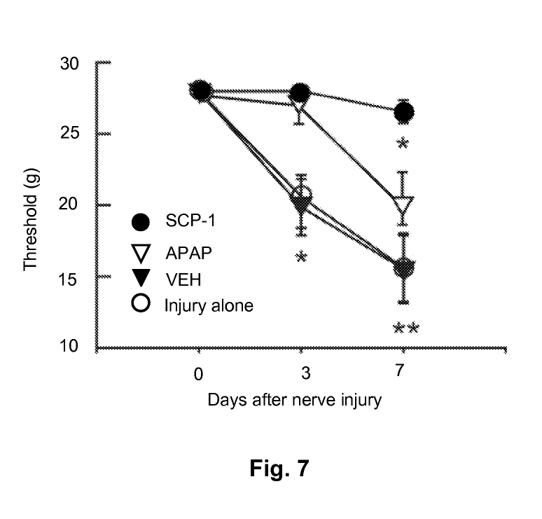

Pre-emptive intrathecal treatment with SCP-1, APAP, or control (vehicle) was given immediately prior to CCI in Bennett rats, and then the rats were assayed for behavioral and biochemical changes. FIG. 7 shows the paw withdrawal threshold changes seen at 0, 3, and 7 days after the injury and injection, using the von Frey filament stimulus for tactile allodynia. Tactile allodynia, as indicated by an abnormally low withdrawal threshold, was prevented by the pre-emptive injection with SCP-1 (100 nmol in 10 μl saline). This effect remained for up to 7 days. The rats injected with vehicle (10 μl saline) showed a response similar to the non-injected, injured rats. A significant difference was seen between SCP-1 pretreatment (n=12) and either vehicle (n=8) or injury alone (n=9) rats (*p<0.05 at 3 days and **p<0.01 at 7 days, respectively, using a two-way Student's t test). Injection with acetaminophen (APAP) (100 nmol in 10 μl saline; n=10) showed an effect similar to SCP-1. At 3 days after nerve injury, no difference for rat paw withdrawal thresholds was observed between SCP-1 and acetaminophen treated rats. However, at 7 days the effect of acetaminophen was sharply reduced while the SCP-1 effect remained strong. The difference in paw withdrawal thresholds at 7 days between the SCP-1 rats and the APAP rats was significant (*p<0.05, one-way ANOVA, followed by Tukey-Kramer Multiple Comparison test). In addition, the difference in paw withdrawal thresholds at 3 days was significant between APAP and injury alone rats (*p<0.05).

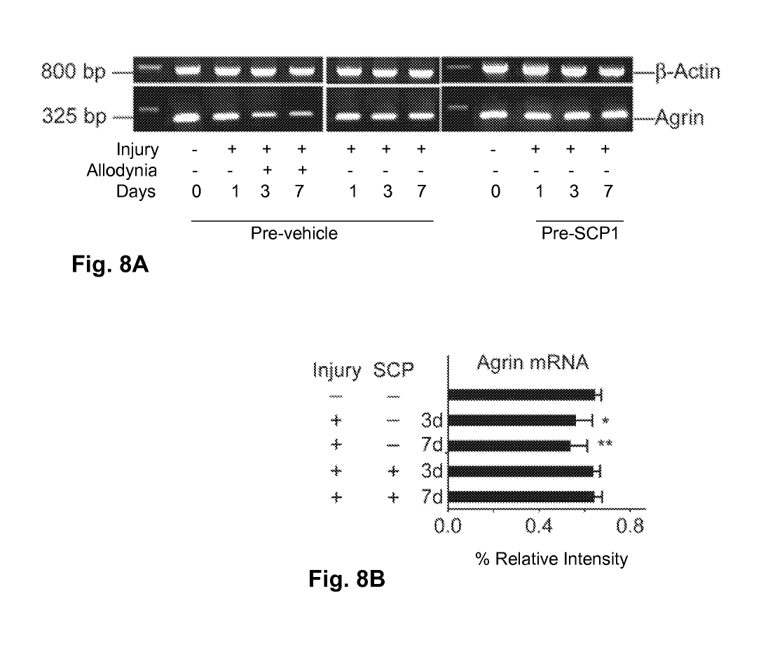

FIG. 8A shows the results of RT-PCR using C-terminal agrin primers and β-actin as a control. As shown in 8A, the rats pre-emptively injected with vehicle showed a decrease in agrin mRNA, while rats injected with SCP-1 did not show a decrease in agrin mRNA. FIG. 8B illustrates the relative intensity of the agrin mRNA bands as compared with the β-actin band at both 3 and 7 days post-injury. The band intensity was averaged for each group. Again, as indicated in FIG. 8A, the mRNA band intensity in rats pre-emptively injected with SCP-1 was the same as in the non-injured rats. However, a significant difference is found between rats injected with SCP-1 (n=10) and with vehicle (n=12) at both 3 and 7 day time frame (*p<0.05 using two way Student's t test).

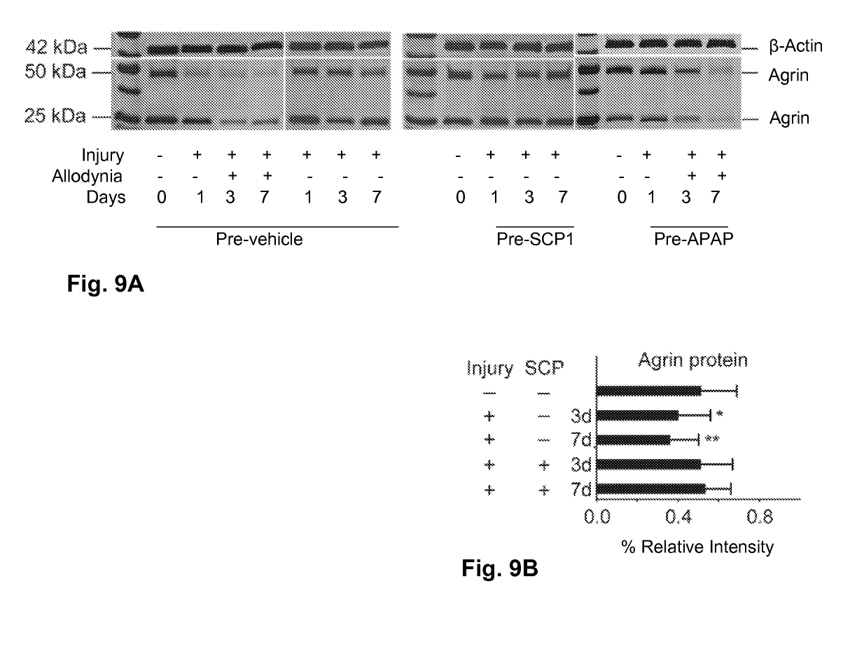

FIG. 9A shows the amount of agrin protein as measured using a Western Blot analysis as described in Example 1. Consistent with data for agrin mRNA, the agrin protein decreased in rats treated with vehicle just prior to injury at both 3 and 7 days. However, agrin protein did not decrease in the rats injected preemptively with SCP-1. Pre-emptive injection with APAP did not prevent the drop in agrin protein at both time points. FIG. 9B illustrates the relative intensity of the agrin protein bands as compared with the β-actin band at both 3 and 7 days. The band intensity was averaged for each group. As shown in FIG. 9B, rats injected pre-emptively with SCP-1 (n=10) had an average band intensity significantly higher than rats injected with vehicle (n=14) at both 3 and 7 day time periods (*p<0.05 and **p<0.05, respectively, using two way Student's t test). Pre-emptive injection with APAP resulted in a band intensity similar to vehicle injection. (Data not shown).

Thus pre-emptive SCP-1 suppressed NP development in the rat model as tested above using a set of von Frey filaments, and inhibited down-regulation of agrin due to the injury. These data support that SCP-1 has inhibitory effects on spinal excitability.

### EXAMPLE 7

### Up-Regulation of Agrin in the Dorsal Horn by Pre-Emptive Treatment with SCP-1

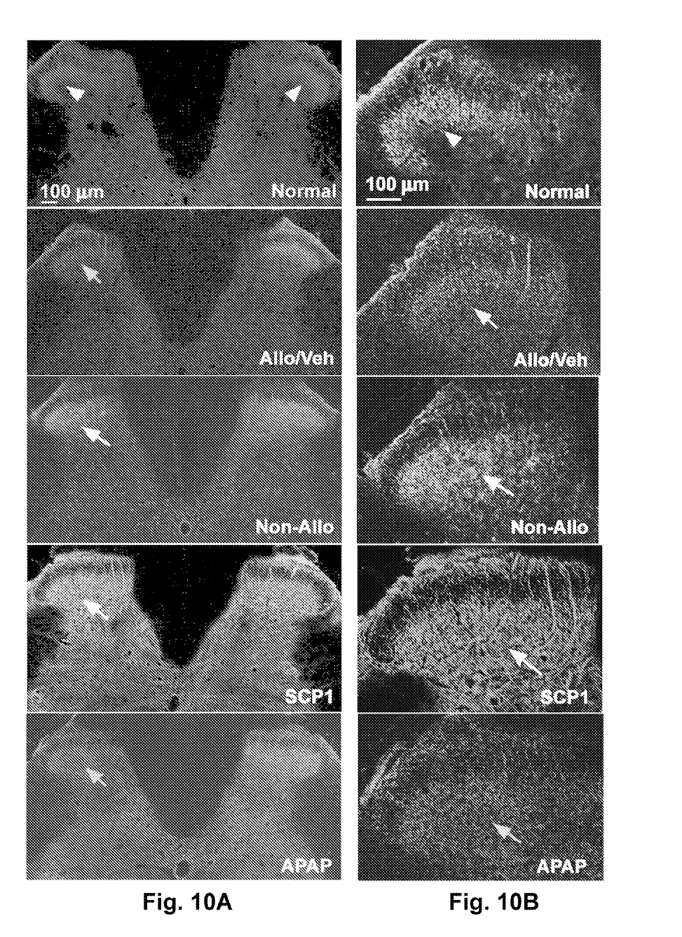

Immunostaining was used to visualize the location and amount of agrin in the spinal cord. Rats (normal, allodynic, non-allodynic, and allodynic animals pre-treated with SCP-1, APAP, or vehicle prior to the injury) were perfused with 4% paraformaldehyde at 7-day post-injury, and dissected for the L4-5 spinal cord (n=4, for each group). These spinal cord samples were cut at 20 μm with a cryostat, and stained with agrin antibody as described in Examples 1 and 4. FIG. 10A shows the results as seen in the dorsal horns. In normal rats, agrin immunoreactivity was bilaterally symmetrical in the dorsal horn, mostly concentrated on lamina I-III. (FIG. 10A, top panel “Normal”). In allodynic rats (also in vehicle- and APAP-treated allodynic rats), agrin immunoreactivity appeared to be much lower in lamina I-III of the ipsilateral dorsal horn to the injury than in that of the contralateral dorsal horn. Also, agrin immunoreactivity in the dorsal horn was much lower in the allodynic rats than in the normal rats. (FIG. 10A, compare Normal to Allo/Veh). The lucent space between lamina I and II was larger in the ipsilateral than in the contralateral dorsal horn in the allodynic rats than in the normal rats. (FIG. 10A) In vehicle- and APAP-treated rats, agrin immunoreactivity in the dorsal horn decreased in the same manner as that in allodynic rats. (FIG. 10A, Compare Normal, Allo/Veh, and APAP. However, in non-allodynic rats, agrin immunoreactivity did not decrease, but was similar to normal rats. (FIG. 10A, compare Normal to Non-Allo). Agrin immunoreactivity in the dorsal horn was elevated by SCP-1 treatment to a level even higher than in normal rats. (FIG. 10A, compare Normal to SCP1). The agrin immunoreactivity in the lucent space between lamina I and II on the injured side was restored by SCP-1 to the level seen in normal rats.

FIG. 10B shows an enlargement of the left dorsal horns from FIG. 10A. The enlarged version visualizes the changes reported above. Agrin immunoreactivity was markedly reduced in lamina I-III in allodynic, vehicle-, and APAP-treated rats, while SCP-1 increased the immunoreactivity in the dorsal horn over that seen in normal rats. (FIG. 10B).

Thus clear differences in agrin intensity were observed among normal, SCP-1 treated, and allodynic vehicle-treated dorsal horns. These results further support the Western blot and behavioral results that agrin is a key molecule in NP modulation.

### EXAMPLE 8

### Modulation of Allodynia and Hyperalgesia in Neuropathic Rats

A blind test was designed for intrathecal drug evaluations. Five days after CCI, rats were subjected to tactile allodynia tests with von Frey filaments. If a filament corresponding to 8 grams or less evoked a withdrawal response on the rat paw (at least 3 responses out of 5 stimuli), the value was designated as the allodynia threshold. A PE10 catheter was installed via L5 and L6 vertebra in rats on day 6 post-injury as described in Example 1 for injecting either SCP-1, SCP-M1 (also called JMM), acetaminophen (APAP), or vehicle. Either a drug (100 nmol in 10 μl saline) or vehicle (only 10 μl saline) was pre-warmed and intrathecally injected into the allodynic rats. Then the rats were assayed for changes in allodynic and hyperalgesic behavior, agrin gene expression, and agrin protein changes. In addition, distribution and amount of agrin in the dorsal horn were visualized using immunostaining of L5 as reported above in Example 7.

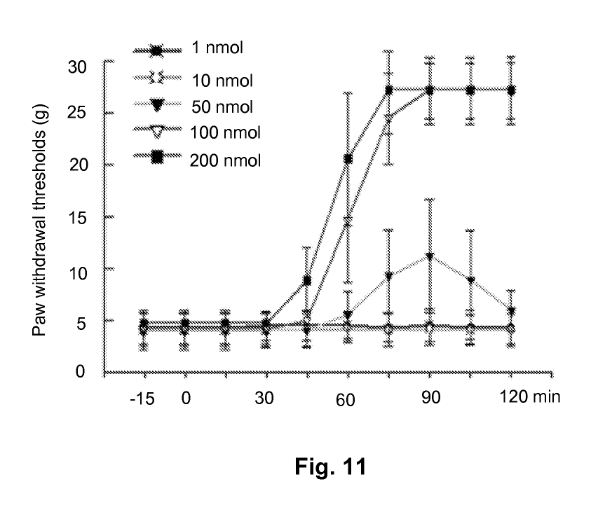

FIG. 11 shows that SCP-1 elevates the paw withdrawal threshold for tactile allodynia in a dose-dependent manner. Allodynic rats were injected with various concentrations of SCP-1 (1 nmol, 10 nmol, 50 nmol, 100 nmol, and 200 nmol) and were then tested for paw withdrawal using the von Frey filaments. As shown in FIG. 11, concentrations of 100 and 200 nmol SCP-1 suppressed the tactile allodynia with 30 to 45 min latency. Concentrations of 1 and 10 nmol SCP-1 did not affect tactile allodynia, while 50 nmol had only a mild effect.

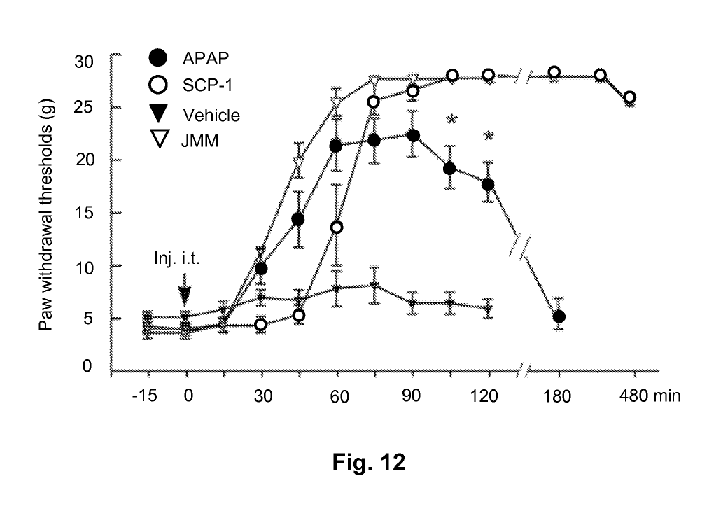

FIG. 12 shows the paw-withdrawal threshold changes in allodynic rats that were induced by the intrathecal injection of various drugs. The paw-withdrawal experiments were as described above in Example 1, and were assessed using von Frey filaments every 15 min. Injection of vehicle (10 μl, n=6) did not affect the low withdrawal thresholds that are indicative of tactile allodynia. Injection of SCP-1 (100 nmol in 10 μl, n=10) raised the withdrawal threshold to normal values about an hour after injection, an effect that lasted for up to 8 hr. (FIG. 12). As shown in FIG. 12, injection of JMM (SCP-M1, 100 nmol in 10 μl, n=8) resulted in a faster response and a faster return to normal values for withdrawal thresholds than was seen in SCP-1. Injection with APAP (100 nmol in 10 μl, n=9) had a less robust effect on the withdrawal threshold, and the effect was of short duration. (FIG. 12). The withdrawal thresholds increased to 28±2.4 g from the basal threshold of 4.6±1.1 g at 90 min after SCP-1 injection. At 2 hr post-injection, the withdrawal thresholds of rats given SCP-1 were still 27.3±2.5 g. APAP administered at the same dose and volume produced a mild threshold increase up to a maximum of about 22 g. The effect of APAP on the thresholds was faster than that of SCP-1, but began to diminish earlier at 90 min after injection. Vehicle (saline) injected intrathecally in the same volume had little effect on withdrawal thresholds. The difference between either SCP-1 or SCP-M1 (JMM) and APAP was significant, as was the difference between APAP and vehicle (*p<0.05, one-way ANOVA, followed by the Tukey-Kramer Multiple Comparison test).

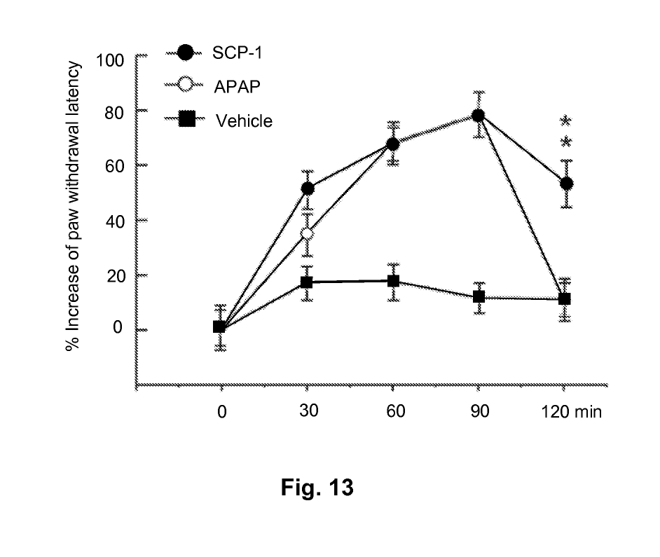

In addition, thermal hyperalgesia (hot-plate) tests were run on the rats given the above three solutions. The results are shown in FIG. 13. SCP-1 in a dose of 100 nmol in 10 μl suppressed thermal hyperalgesia (as measured by increased paw-withdrawal latency by 80% in the CCI (Bennett) rat model. APAP (n=8) had a similar effect on latency. However, at 120 min post injection, the effect of APAP dropped to the initial latency value, while the effect of SCP-1 was still apparent. The difference between SCP-1 and APAP at the 120 min check-point was significant (**, P<0.01, One-way ANOVA, followed by Tukey-Kramer Multiple Comparison-test). The saline vehicle did not influence latency. These results are an additional demonstration that SCP-1 has a robust suppressive effect on neuropathic pain as measured by tactile allodynia and thermal hyperalgesia responses.

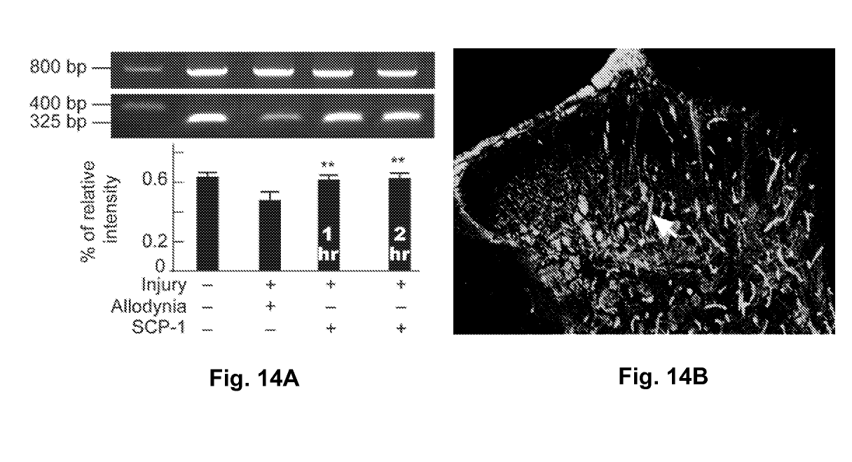

FIG. 14A (top panel) shows the amount of agrin mRNA as measured by RT-PCR at one- and two-hour post injection of SCP-1. The SCP-1 injection was shown to suppress tactile allodynia as reported above. The top row in FIG. 14A shows the bands for β-actin, while the lower band shows the agrin mRNA as measured with the C-terminal agrin primers. Agrin mRNA was increased by SCP-1 injection at both one- and two-hour sample times. In contrast, vehicle injection did not change the amount of agrin mRNA. FIG. 14A (bottom graph) illustrates the relative intensity of the agrin mRNA bands as compared to the β-actin bands. The intensity of the mRNA band was significantly greater than the mRNA band of the rats injected with vehicle (n=8, **p<0.01, two way Student's t test).

FIG. 14B is a micrograph of the dorsal horn of a spinal cord after immunostaining with agrin antibody from an allodynic rat injected with SCP-1, and sacrificed 1.5 h after injection. The arrow in FIG. 14B indicates the growth of an agrin fiber.

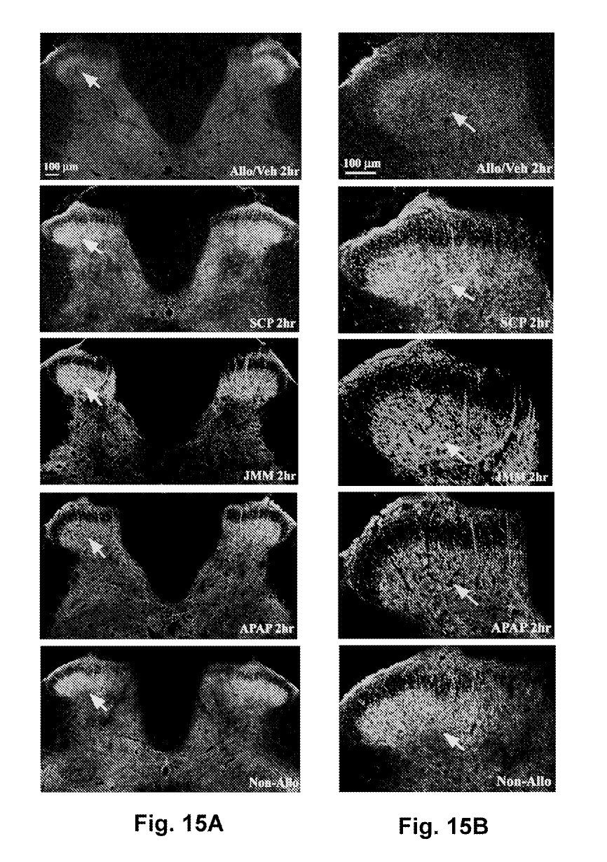

Immunostaining was again used to visualize the location and amount of agrin in the dorsal horn of the spinal cord. Two hr after injecting the rats with SCP-1, SCP-M1 (JMM), APAP and vehicle, the rats were perfused with 4% paraformaldehyde and dissected for the L4-5 spinal cord. These spinal cord samples were cut at 20 μm with a cryostat, and stained with agrin antibody as described in Example 1. FIG. 15A shows the results as seen in the dorsal horn. Agrin immunoreactivity was greatly increased in lamina I-IV of the dorsal horns in rats injected with SCP-1 and SCP-M1 (JMM). No agrin increase was seen in the vehicle- or APAP injected rats, In addition, the non-allodynic rats had a higher level of agrin than did the vehicle-injected, allodynic rats. Compared to non-allodynic rats, agrin immunoreactivity was obviously decreased in the dorsal horn of allodynic rats. FIG. 15B is just an enlarged version of the left dorsal horn from FIG. 15A. The agrin protein increases in the dorsal horn were parallel to the suppression of tactile allodynia induced by intrathecal SCP-1 injection in the behavioral experiments. (see FIGS. 11, 12, and 13).

All of the above results indicate that agrin plays an important role in allodynic suppression, and that an increase in agrin can reverse the allodynic response. These results also show that both SCP-1 and SCP-M1 are effective agents that can cause an upregulation in agrin expression in allodynic rats.

### EXAMPLE 9

### Agrin siRNA Blocked the Effect of SCP-1 on Tactile Allodynia

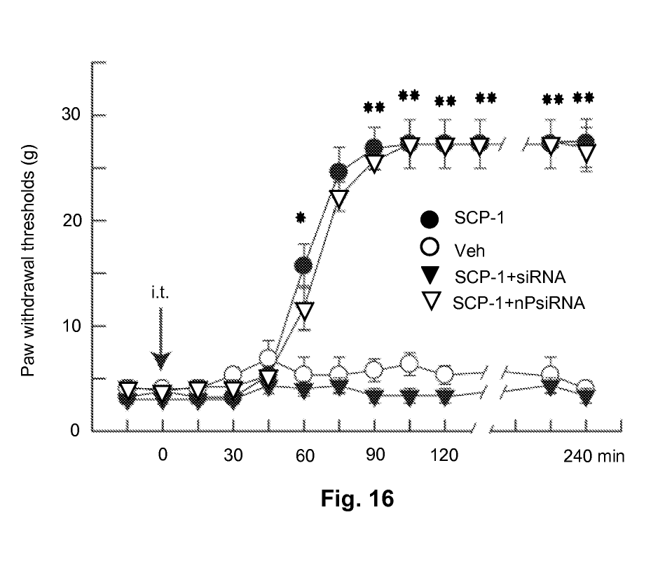

Small interference RNA (siRNA) of a gene suppresses the gene's physical function in living cells. Rats were injected 7 days after CCI with vehicle, SCP-1, SCP-1 plus agrin siRNA, or SCP-1 plus agrin nFsiRNA (non-functional siRNA), and then the paw withdrawal threshold was measured with von Frey filaments for tactile allodynia. Injection with vehicle did not elevate the low withdrawal threshold that is characteristic of allodynic rats. (FIG. 16, Veh) SCP-1 normalized the withdrawal threshold in about one hour after injection, and the suppressive effect lasted for at least 4 hr. (FIG. 16, SCP-1) When C-terminal siRNA (100 nM) was given intrathecally at 5 μl/hour continuously after the bolus injection of SCP-1, the C-terminal agrin siRNA antagonized the SCP-1 and inhibited the effect on withdrawal threshold. However, when non-functional agrin C-terminal siRNA (nFsiRNA) was given, the SCP-1 effect was not blocked and the threshold was elevated to normal levels. These results further confirm that the effect of SCP-1 in suppressing allodynia is mediated via changes in agrin.

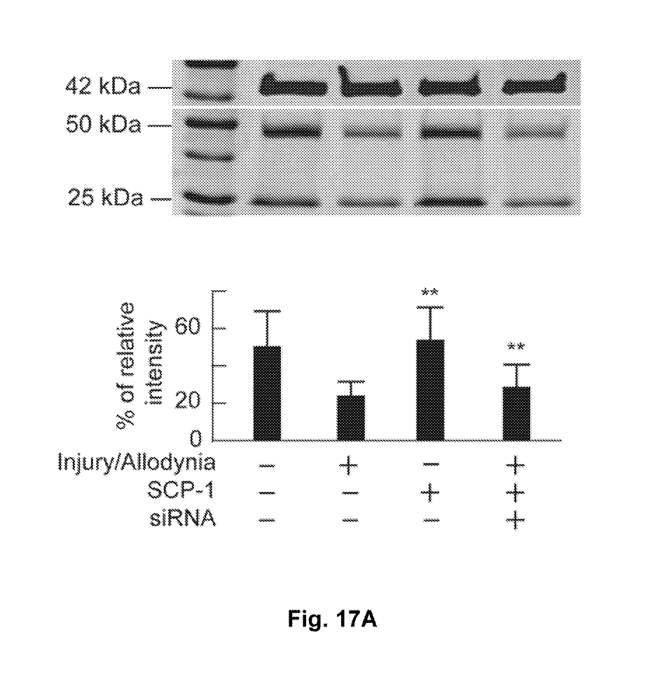

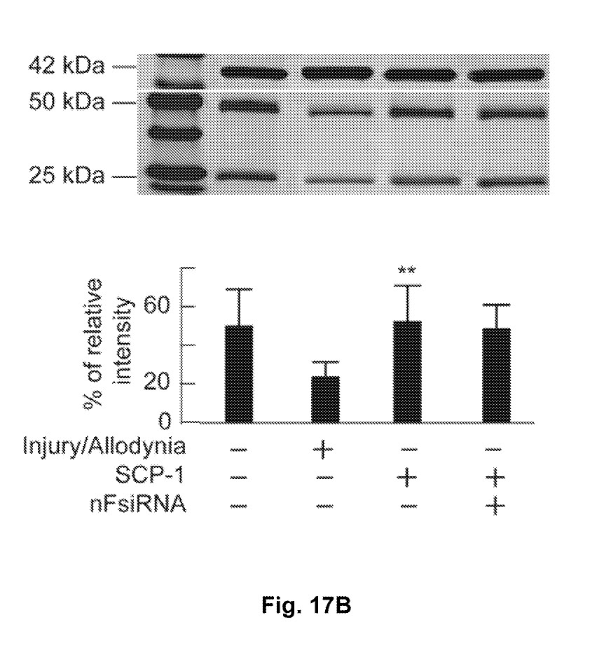

Western blot analysis of rats treated as above support the same conclusion. FIG. 17A shows the results of Western blot analysis for agrin protein expression produced by injections of SCP-1 and agrin C-terminal siRNA plus SCP-1, using β-actin as a control, and rats sacrificed at 2 h post-injection. The top panel shows the Western blot bands, and the bottom graph shows the relative intensity of the agrin bands as compared to the β-actin bands. These results confirm that agrin protein level was up-regulated to a normal level at 2 h post-SCP-1 injection. The results for agrin protein 4 h post-SCP-1 injection were the same (Data not shown). When siRNA was injected with the SCP-1, the agrin did not return to normal levels, but was similar to allodynic rats. (FIG. 17A) There were significant differences between (1) normal and allodynic rats, (2) allodynic and SCP-1-treated allodynic rats, and (3) SCP-1-treated allodynic rats and siRNA plus SCP-1-treated allodynic rats (**, p<0.01 (n=8), One-way ANOVA, followed by Tukey-Kramer Multiple Comparison test).

FIG. 17B shows the results of Western blot analysis for agrin protein produced by injections of SCP-1 and SCP-1 plus non-functional agrin C-terminal siRNA (nFsiRNA). The results are presented as relative band intensity as compared to β-actin, as described above. As shown in FIG. 17B, the nFsiRNA did not affect the agrin protein up-regulation produced by SCP-1. There was no significant difference between the agrin band intensity produced by injection of either SCP-1 or nFsiRNA plus SCP-1. (n=8). These results support that agrin is a key molecule in NP modulation, and that SCP-1 suppresses tactile allodynia by elevating agrin protein in the dorsal horn.

### EXAMPLE 10

### Injection with SCP-1 and SCP-M1 Causes Changes in the Dorsal Horn of Phosphorylated NR1 Subunits of the NMDA Receptor

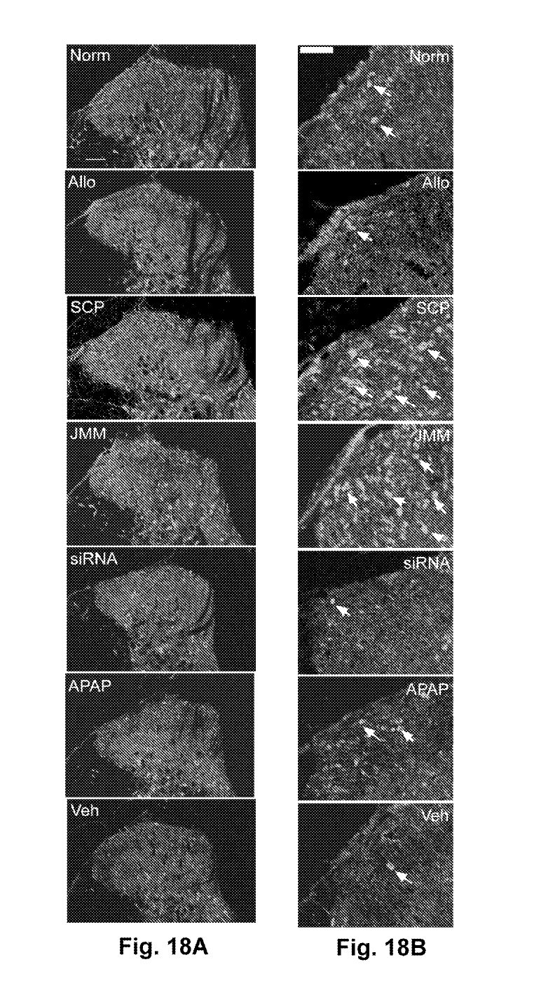

It has been shown that when the NR1 subunit of the NMDA receptor is phosphorylated, then the NMDA receptor excitability is suppressed. This suppression would cause a reduction in pain. An experiment was conducted to look at changes in the number of phosphorylated NR1 subunits (p-NR1) of the NMDA receptor in the dorsal horn induced by intrathecal injection of various drugs in allodynic rats. An antibody that targeted the serine residue 897/896 phosphorylation of NMDA receptor NR1 subunit (p-NR1) (Upstate, Charlottesville, Va.) was used to label the dorsal horn, similar to methods described above. The rats were injected with 100 nmol of SCP-1, SCP-M1 (JMM), APAP, vehicle, and SCP-1 plus agrin siRNA, and then sacrificed either at 2 h or 4 h later. The results are shown in FIGS. 18A and 18B, with 18B being an enlargement of the dorsal horn in FIG. 18A. In the dorsal horn from a normal rat, only a few p-NR1 positive neurons were observed, while even less p-NR1 positive neurons were seen in the allodynic rat. Vehicle injection did not change the number of p-NR1 positive neurons. In contrast, both SCP-1 and SCP-M1 (JMM) up-regulated the number of p-NR1 positive neurons in the dorsal horn. When agrin C-terminal siRNA was given together with SCP-1, the up-regulation of p-NR1 positive neurons by SCP-1 was not seen. Intrathecal injection of APAP did not induce significant up-regulation of p-NR1 positive neurons in the dorsal horn. FIG. 18B shows the enlarged dorsal horn from FIG. 18A. It shows more clearly the change in p-NR1 positive neurons.

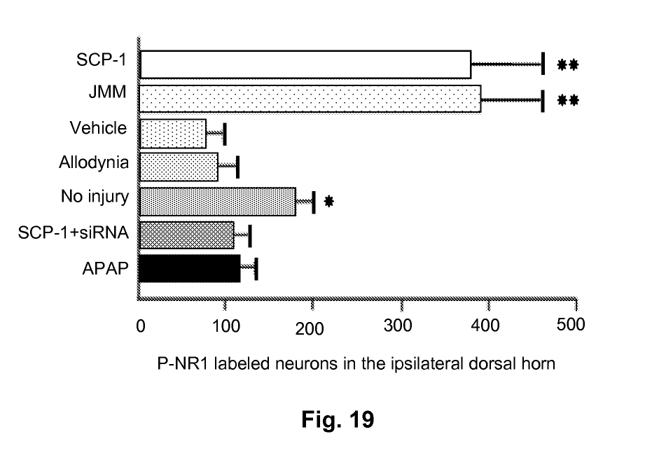

FIG. 19 illustrates the number of p-NR1 positive neurons in the dorsal horn in the experiment described above. The bars represent the mean±SD for the different treatments. In the SCP-1 and SCP-M1 (JMM) treated rats, the number of p-NR1 positive neurons were 373±86 and 388±74, respectively. This is in strong contrast to the numbers in vehicle treated rats (74±22), in allodynic rats (87±27), and in no injury rats (175±24). When agrin siRNA was applied together with SCP-1, the p-NR1 positive neurons dropped to 102±22, suggesting that agrin siRNA inhibited the phosphorylation at NR1 serine residue sites. APAP did not induce significant increase in p-NR1 positive neurons (111±24). There is a significant difference between the SCP-1-treated rats and the vehicle or allodynic rats. (**p<0.01). Equally significant is the difference between SCP-M1 (JMM)-treated and vehicle or allodynic rats. There is also a significant difference in p-NR1 positive neurons between normal and allodynic rats (*p<0.05, One-way ANOVA, followed by Mann-Whitney test).

These results support that the agrin protein increase in the dorsal horn that is induced by SCP-1 suppresses the tactile allodynia at least in part through the serine phosphorylation sites of NR1 subunit where agrin activates serine kinase to depress NMDA receptor excitability.

Initial experiments have been conducted applying a C-terminal agrin fragment to dorsal spinal cell cultures. The agrin segment was rat agrin (ala 1153-pro 1948), a 90 kDa C-terminal fragment, isoform C—Ag_{3,4,8}, purchased from R&D Systems, Inc. (Catalog no. 550-AG/CF, Minneapolis, Minn.). In just 15 min after applying the agrin fragment, an increase in phosphorylated NR1 was seen using fluorescence markers. (Data not shown) This indicated that agrin acts in part by increasing the phosphorylation of NR1. Based on this data and the above data indicating the importance of C-terminal agrin fragments, it is also believed that the C-terminal agrin fragment of size about 70 kDa^{[85]} would also be effective in preventing or alleviating neuropathic pain.

Miscellaneous

The term “agrin” used herein and in the claims refers to the peptide agrin, the approximately 200 kDa polypeptide backbone or the approximately 400 kDa heparin sulfate proteoglycan. The term “C-terminal agrin fragment” refers to a segment of agrin that contains the C-terminus, e.g., fragments with a size of approximately 95 kDa, 70 kDa, 50 kDa or 25 kDa, and that is decreased in neuropathic pain models. These fragments can be from various agrin variants, with different amino acids at the known insertions sites of X, Y, and Z. The term “homologs” refers to polypeptides in which one or more amino acids have been replaced by different amino acids, such that the resulting polypeptide is at least 75% homologous, and preferably 85% homologous, to the basic sequence as, for example, the sequence of agrin or one of the fragments of the C-terminal of agrin, and where the variant polypeptide retains the activity of the basic polypeptide, for example, agrin or one of the fragments of the C-terminal of agrin. Homology is defined as the percentage number of amino acids that are identical or constitute conservative substitutions. Conservative substitutions of amino acids are well known. The term “homolog” includes synthetically generated polypeptides, as well as naturally occurring allelic variants, for example, the known 20 variants of agrin^{[29,35,36]}.

The term “derivative” refers to a polypeptide that has been derived from the basic sequence by modification, including amino acid deletions or additions to polypeptides or variants and modification to side chains, where the derivative retains the activity of the basic protein, for example, agrin or a C-terminal agrin fragment. The resulting derivative will retain at least 75% homology and preferably 85% homology with the basic sequence of the original polypeptide. The derivative will also exhibit a qualitatively similar effect to the unmodified polypeptide.

The term “therapeutically effective amount” as used herein refers to an administered amount of agrin, a C-terminal agrin fragment, or of a drug that increases the amount of agrin in the central nervous system sufficient to prevent or to ameliorate neuropathic pain in a mammal to a statistically significant degree (p<0.05). The term “exogenous” refers to a compound that is derived or developed outside the body, and thus must be administered to the subject. The term “endogenous” refers to a compound that originates or develops within an organism. The term “therapeutically effective amount” therefore includes, for example, an amount sufficient to decrease tactile or thermal allodynia/hyperalgesia due to an injury, preferably to reduce it by at least 50%, and more preferably to reduce it by at least 90%. The dosage ranges for the administration of agrin or a drug (e.g., SCP-1 or SCP-M1 (JMM)) are those that produce the desired effect. Generally, the dosage will vary with the age, weight, condition, sex of the patient, type of injury, and the degree of neuropathic pain. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the level of neuropathic pain by methods well known to those in the field. Moreover, agrin or a drug to increase agrin can be applied in pharmaceutically acceptable carriers known in the art. The application can be oral, by injection, or topical, but the preferred method is intrathecal injection.

The present invention provides a method of preventing, treating, or ameliorating neuropathic pain in a mammal, comprising administering to a mammal pre-surgery, close to the time of injury or surgery, or post-surgery or injury, a therapeutically effective amount of agrin, a C-terminal agrin fragment, or a drug that causes an increase in agrin gene expression. The term “ameliorate” refers to a decrease or lessening of the symptoms or signs of the disorder being treated.

### REFERENCES CITED

1. Woolf, C. J. and Max, M. B., Mechanism-based pain diagnosis: issues for analgesic drug development. Anesthesiology. 2001; 95: 241-249.
2. Dworkin, R. H., Backonja, M., Rowbotham, M. C., Allen, R. R., Argoff, C. R., Bennett, G. J., Bushnell, M. C., Farrar, J. T., Galer, B. S., Haythornthwaite, J. A., Hewitt, D. J., Loeser, J. D., Max, M. B., Saltarelli, M., Schmader, K. E., Stein, C., Thompson, D., Turk, D. C., Wallace, M. S., Watkins, L. R., and Weinstein, S. M., Advances in neuropathic pain: diagnosis, mechanisms, and treatment recommendations. Arch. Neurol. 2003; 60: 1524-1534.
3. Woolf, C. J. and Salter, M. W., Neuronal plasticity: increasing the gain in pain. Science, 2000; 288: 1765-1769.
4. Pertovaara, A., Collateral sprouting of nociceptive C-fibers after cut or capsaicin treatment of the sciatic nerve in adult rats. Neurosci. Lett., 1988; 90: 248-253.
5. Tal, M. and Bennett, G. J., Extra-territorial pain in rats with a peripheral mononeuropathy: mechano-hyperalgesia and mechano-allodynia in the territory of an uninjured nerve. Pain, 1994; 57: 375-382.
6. McLachlan, E. M., Janig, W., Devor, M., and Michaelis, M., Peripheral nerve injury triggers noradrenergic sprouting within dorsal root ganglia. Nature, 1993; 363: 543-546.
7. Na, H. S., Leem, J. W., and Chung, J. M., Abnormalities of mechanoreceptors in a rat model of neuropathic pain: possible involvement in mediating mechanical allodynia. J. Neurophysiol., 1993; 70: 522-528.
8. Devor, M., The pathophysiology of damaged peripheral nerve. 1994; 3rd: 70-100.
9. Chaplan, S. R., Guo, H. Q., Lee, D. H., Luo, L., Liu, C., Kuei, C., Velumian, A. A., Butler, M. P., Brown, S. M., and Dubin, A. E., Neuronal hyperpolarization-activated pacemaker channels drive neuropathic pain. J. Neurosci., 2003; 23: 1169-1178.
10. Coderre, T. J., Katz, J., Vaccarino, A. L., and Melzack, R., Contribution of central neuroplasticity to pathological pain: review of clinical and experimental evidence. Pain, 1993; 52: 259-285.
11. Hokfelt, T., Zhang, X., and Wiesenfeld-Hallin, Z., Messenger plasticity in primary sensory neurons following axotomy and its functional implications. Trends Neurosci., 1994; 17: 22-30.
12. Cui, J. G., O'Connor, W. T., Ungerstedt, U., Linderoth, B., and Meyerson, B. A., Spinal cord stimulation attenuates augmented dorsal horn release of excitatory amino acids in mononeuropathy via a GABAergic mechanism. Pain, 1997; 73: 87-95.
13. Ji, R. R. and Woolf, C. J., Neuronal plasticity and signal transduction in nociceptive neurons: implications for the initiation and maintenance of pathological pain. Neurobiol. Dis., 2001; 8: 1-10.
14. Chen, L. and Huang, L. Y., Protein kinase C reduces Mg2+ block of NMDA-receptor channels as a mechanism of modulation. Nature, 1992; 356: 521-523.
15. Mao, J., Price, D. D., Hayes, R. L., Lu, J., and Mayer, D. J., Differential roles of NMDA and non-NMDA receptor activation in induction and maintenance of thermal hyperalgesia in rats with painful peripheral mononeuropathy. Brain Res., 1992; 598: 271-278.
16. McMahon, S. B. and Wall, P. D., Receptive fields of rat lamina 1 projection cells move to incorporate a nearby region of injury. Pain, 1984; 19: 235-247.
17. Woolf, C. J. and King, A. E., Dynamic alterations in the cutaneous mechanoreceptive fields of dorsal horn neurons in the rat spinal cord. J. Neurosci., 1990; 10: 2717-2726.
18. Opree, A. and Kress, M., Involvement of the proinflammatory cytokines tumor necrosis factor-alpha, IL-1 beta, and IL-6 but not IL-8 in the development of heat hyperalgesia: effects on heat-evoked calcitonin gene-related peptide release from rat skin. J. Neurosci., 2000; 20: 6289-6293.
19. Cui, J. G., Holmin, S., Mathiesen, T., Meyerson, B. A., and Linderoth, B., Possible role of inflammatory mediators in tactile hypersensitivity in rat models of mononeuropathy.
Pain, 2000; 88: 239-248.
20. Laughlin, T. M., Bethea, J. R., Yezierski, R. P., and Wilcox, G. L., Cytokine involvement in dynorphin-induced allodynia. Pain, 2000; 84: 159-167.
21. Samad, T. A., Moore, K. A., Sapirstein, A., Billet, S., Allchome, A., Poole, S., Bonventre, J. V., and Woolf, C. J., Interleukin-1 beta-mediated induction of Cox-2 in the CNS contributes to inflammatory pain hypersensitivity. Nature, 2001; 410: 471-475.
22. Bazan, N. G. and Flower, R. J., Medicine: lipid signals in pain control. Nature, 2002; 420: 135-138.
23. Shamash, S., Reichert, F., and Rotshenker, S., The cytokine network of Wallerian degeneration: tumor necrosis factor-alpha, interleukin-1 alpha, and interleukin-1 beta. J. Neurosci., 2002; 22: 3052-3060.
24. Lin, Q., Peng, Y. B., and Willis, W. D., Possible role of protein kinase C in the sensitization of primate spinothalamic tract neurons. J. Neurosci., 1996; 16: 3026-3034.
25. Mao, J., Price, D. D., Mayer, D. J., and Hayes, R. L., Pain-related increases in spinal cord membrane-bound protein kinase C following peripheral nerve injury. Brain Res., 1992; 588: 144-149.
26. Dina, O. A., Chen, X., Reichling, D., and Levine, J. D., Role of protein kinase Cepsilon and protein kinase A in a model of paclitaxel-induced painful peripheral neuropathy in the rat. Neuroscience, 2001; 108: 507-515.
27. Kryger, G. S., Kryger, Z., Zhang, F., Shelton, D. L., Lineaweaver, W. C., and Buncke, H. J., Nerve growth factor inhibition prevents traumatic neuroma formation in the rat. J. Hand Surg. [Am.], 2001; 26: 635-644.
28. Ji, R. R., Samad, T. A., Jin, S. X., Schmoll, R., and Woolf, C. J., p38 MAPK activation by NGF in primary sensory neurons after inflammation increases TRPV1 levels and maintains heat hyperalgesia. Neuron., 2002; 36: 57-68.
29. Rupp, F., Ozcelik, T., Linial, M., Peterson, K., Francke, U., and Scheller, R., Structure and chromosomal localization of the mammalian agrin gene. J. Neurosci., 1992; 12: 3535-3544.
30. Magill, C., Reist, N. E., Fallon, J. R., Nitkin, R. M., Wallace, B. G., and McMahan, U. J., Agrin. Prog. Brain Res., 1987; 71: 391-396.
31. McMahan, U. J., The agrin hypothesis. Cold Spring Harb. Symp. Quant. Biol., 1990; 55: 407-418.
32. Bowe, M. A. and Fallon, J. R., The role of agrin in synapse formation. Annu. Rev. Neurosci., 1995; 18: 443-462.
33. Gesemann, M., Denzer, A. J., and Ruegg, M. A., Acetylcholine receptor-aggregating activity of agrin isoforms and mapping of the active site. J. Cell Biol., 1995; 128: 625-636.
34. Hoover, C. L., Hilgenberg, L. G., and Smith, M. A., The COOH-terminal domain of agrin signals via a synaptic receptor in central nervous system neurons. J. Cell Biol., 2003; 161: 923-932.
35. Ferns, M., Hoch, W., Campanelli, J. T., Rupp, F., Hall, Z. W., and Scheller, R. H., RNA splicing regulates agrin-mediated acetylcholine receptor clustering activity on cultured myotubes. Neuron., 1992; 8: 1079-1086.
36. Ferns, M. J., Campanelli, J. T., Hoch, W., Scheller, R. H., and Hall, Z., The ability of agrin to cluster AChRs depends on alternative splicing and on cell surface proteoglycans. Neuron., 1993; 11: 491-502.
37. Cohen, N. A., Kaufmann, W. E., Worley, P. F., and Rupp, F., Expression of agrin in the developing and adult rat brain. Neuroscience, 1997; 76: 581-596.
38. Campanelli, J. T., Hoch, W., Rupp, F., Kreiner, T., and Scheller, R. H., Agrin mediates cell contact-induced acetylcholine receptor clustering. Cell, 1991; 67: 909-916.
39. Mejat, A., Ravel-Chapuis, A., Vandromme, M., and Schaeffer, L., Synapse-specific gene expression at the neuromuscular junction. Ann. N.Y. Acad. Sci., 2003; 998: 53-65.
40. Bezakova, G. and Ruegg, M. A., New insights into the roles of agrin. Nat. Rev. Mol. Cell Biol., 2003; 4: 295-308.
41. Kroger, S, and Schroder, J. E., Agrin in the developing CNS: new roles for a synapse organizer. News Physiol Sci., 2002; 17: 207-212.
42. Bose, C. M., Qiu, D., Bergamaschi, A., Gravante, B., Bossi, M., Villa, A., Rupp, F., and Malgaroli, A., Agrin controls synaptic differentiation in hippocampal neurons. J. Neurosci., 2000; 20: 9086-9095.
43. Mantych, K. B. and Ferreira, A., Agrin differentially regulates the rates of axonal and dendritic elongation in cultured hippocampal neurons. J. Neurosci., 2001; 21: 6802-6809.
44. Bixby, J. L., Baerwald-De la Torre, K., Wang, C., Rathjen, F. G., and Ruegg, M. A., A neuronal inhibitory domain in the N-terminal half of agrin. J. Neurobiol., 2002; 50: 164-179.
45. Serpinskaya, A. S., Feng, G., Sanes, J. R., and Craig, A. M., Synapse formation by hippocampal neurons from agrin-deficient mice. Dev. Biol., 1999; 205: 65-78.
46. Nastuk, M. A., Lieth, E., Ma, J. Y., Cardasis, C. A., Moynihan, E. B., McKechnie, B. A., and Fallon, J. R., The putative agrin receptor binds ligand in a calcium-dependent manner and aggregates during agrin-induced acetylcholine receptor clustering. Neuron, 1991; 7: 807-818.
47. Wells, D. G., McKechnie, B. A., Kelkar, S., and Fallon, J. R., Neurotrophins regulate agrin-induced postsynaptic differentiation. Proc. Natl. Acad. Sci. U.S.A., 1999; 96: 1112-1117.
48. Jung, Kim M., Cotman, S. L., Halfter, W., and Cole, G. J., The heparan sulfate proteoglycan agrin modulates neurite outgrowth mediated by FGF-2. J. Neurobiol., 2003; 55: 261-277.
49. Halfter, W., Schurer, B., Yip, J., Yip, L., Tsen, G., Lee, J. A., and Cole, G. J., Distribution and substrate properties of agrin, a heparan sulfate proteoglycan of developing axonal pathways. J. Comp Neurol., 1997; 383: 1-17.
50. Hilgenberg, L. G., Ho, K. D., Lee, D., O'Dowd, D. K., and Smith, M. A., Agrin regulates neuronal responses to excitatory neurotransmitters in vitro and in vivo. Mol. Cell Neurosci., 2002; 19: 97-110.
51. O'Connor, L. T., Lauterborn, J. C., Smith, M. A., and Gall, C. M., Expression of agrin mRNA is altered following seizures in adult rat brain. Brain Res. Mol. Brain Res., 1995; 33: 277-287.
52. Thomas, W. S., Jacob, M. H., O'Dowd, D. K., and Smith, M. A., Agrin gene expression in ciliary ganglion neurons following preganglionic denervation and postganglionic axotomy. Dev. Biol., 1995; 168: 662-669.
53. Parsons, C. G., NMDA receptors as targets for drug action in neuropathic pain. Eur. J. Pharmacol., 2001; 429: 71-78.
54. McBain, C. J. and Mayer, M. L., N-methyl-D-aspartic acid receptor structure and function. Physiol Rev., 1994; 74: 723-760.
55. Krupp, J. J., Vissel, B., Heinemann, S. F., and Westbrook, G. L., N-terminal domains in the NR2 subunit control desensitization of NMDA receptors. Neuron, 1998; 20: 317-327.
56. Yamakura, T. and Shimoji, K., Subunit- and site-specific pharmacology of the NMDA receptor channel. Prog. Neurobiol., 1999; 59: 279-298.
57. McMahon, S. B., Lewin, G. R., and Wall, P. D., Central hyperexcitability triggered by noxious inputs. Curr. Opin. Neurobiol., 1993; 3: 602-610.
58. Broman, J., Neurotransmitters in subcortical somatosensory pathways. Anat. Embryol. (Berl). 1994; 189: 181-214.
59. Cui, J. G., O'Connor, W. T., Ungerstedt, U., Linderoth, B., and Meyerson, B. A., Spinal cord stimulation attenuates augmented dorsal horn release of excitatory amino acids in mononeuropathy via a GABAergic mechanism. Pain, 1997; 73: 87-95.
60. Mayer, M. L., Westbrook, G. L., and Guthrie, P. B., Voltage-dependent block by Mg2+ of NMDA responses in spinal cord neurones. Nature, 1984; 309: 261-263.
61. Qian, A., Antonov, S. M., and Johnson, J. W., Modulation by permeant ions of Mg(2+) inhibition of NMDA-activated whole-cell currents in rat cortical neurons. J. Physiol., 2002; 538: 65-77.
62. Mao, J., Price, D. D., Hayes, R. L., Lu, J., and Mayer, D. J., Differential roles of NMDA and non-NMDA receptor activation in induction and maintenance of thermal hyperalgesia in rats with painful peripheral mononeuropathy. Brain Res., 1992; 598: 271-278.
63. Suzuki, R., Matthews, E. A., and Dickenson, A. H., Comparison of the effects of MK-801, ketamine and memantine on responses of spinal dorsal horn neurones in a rat model of mononeuropathy. Pain, 2001; 91: 101-109.
64. Chaplan, S. R., Malmberg, A. B., and Yaksh, T. L., Efficacy of spinal NMDA receptor antagonism in formalin hyperalgesia and nerve injury evoked allodynia in the rat. J. Pharmacol. Exp. Ther., 1997; 280: 829-838.
65. Taniguchi, K., Shinjo, K., Mizutani, M., Shimada, K., Ishikawa, T., Menniti, F. S., and Nagahisa, A., Antinociceptive activity of CP-101,606, an NMDA receptor NR2B subunit antagonist. Br. J. Pharmacol., 1997; 122: 809-812.
66. Boyce, S., Wyatt, A., Webb, J. K., O'Donnell, R., Mason, G., Rigby, M., Sirinathsinghji, D., Hill, R. G., and Rupniak, N. M., Selective NMDA NR2B antagonists induce antinociception without motor dysfunction: correlation with restricted localisation of NR2B subunit in dorsal horn. Neuropharmacology, 1999; 38: 611-623.
67. Vaccarino, A., Rodriguez de Turco, E. B., Marcheselli, V. L., Paul, D., Alvarez-Builla, J., Sunkel, C., Parkins, N., Heather, S., and Bazan, N. G., SCP-1, a novel derivative of acetaminophen: evaluation of analgesia, toxicity, lethality, and antipyresis, Proceedings of 10^{th} World Congress on Pain, San Diego, Calif., 2002;
68. Marcheselli, V. L., Paul, D., Minor, L., Sunkel, C., Alvarez-Builla, J., and Bazan, N. G., SCP-1, a novel analgesic derivative of acetaminophen, lacks its hepatotoxic properties, Proceedings of 32^{rd} Neuroscience Congress, Orlando, Fla., 2001;
69. Cui, J. G., Lukiw, W. J., Zhao, Y. H., Marcheselli, V. L., and Bazan, N. G., SCP-1, a novel potent analgesic for neuropathic pain, Proceedings of 33^{rd} Neuroscience Congress, New Orleans, La., 2003;
70. Kennedy, S., Wang, D., and Ruvkun, G., A conserved siRNA-degrading RNase negatively regulates RNA interference in C. elegans. Nature, 2004; 427: 645-649.
71. Fire, A., Xu, S., Montgomery, M. K., Kostas, S. A., Driver, S. E., and Mello, C. C., Potent and specific genetic interference by double-stranded RNA in Caenorhabditis elegans. Nature, 1998; 391: 806-811.
72. McManus, M. T. and Sharp, P. A., Gene silencing in mammals by small interfering RNAs. Nat. Rev. Genet., 2002; 3: 737-747.
73. Barclay, J., Patel, S., Dorn, G., Wotherspoon, G., Moffatt, S., Eunson, L., Abdel'al, S., Natt, F., Hall, J., Winter, J., Bevan, S., Wishart, W., Fox, A., and Ganju, P., Functional downregulation of P2X3 receptor subunit in rat sensory neurons reveals a significant role in chronic neuropathic and inflammatory pain. J. Neurosci., 2002; 22: 8139-8147.
74. Dorn, G., Patel, S., Wotherspoon, G., Hemmings-Mieszczak, M., Barclay, J., Natt, F. J., Martin, P., Bevan, S., Fox, A., Ganju, P., Wishart, W., and Hall, J., siRNA relieves chronic neuropathic pain. Nucleic Acids Res., 2004; 32: e49
75. Cui, J. G., Linderoth, B., and Meyerson, B. A., Incidence of mononeuropathy in rats is influenced by pre-emptive alteration of spinal excitability. Eur J Pain, 1997; 1: 53-59.
76. Bennett, G. J. and Xie, Y. K., A peripheral mononeuropathy in rat that produces disorders of pain sensation like those seen in man. Pain, 1988; 33: 87-107.
77. Gazelius, B., Cui, J. G., Svensson, M., Meyerson, B., and Linderoth, B., Photochemically induced ischaemic lesion of the rat sciatic nerve. A novel method providing high incidence of mononeuropathy. Neuroreport, 1996; 7: 2619-2623.
78. Prado, R., Dietrich, W. D., Watson, B. D., Ginsberg, M. D., and Green, B. A., Photochemically induced graded spinal cord infarction. Behavioral, electrophysiological, and morphological correlates. J. Neurosurg., 1987; 67: 745-753.
79. Cui, J. G., Linderoth, B., and Meyerson, B. A., Effects of spinal cord stimulation on touch-evoked allodynia involve GABAergic mechanisms. An experimental study in the mononeuropathic rat. Pain, 1996; 66: 287-295.
80. Chaplan, S. R., Bach, F. W., Pogrel, J. W., Chung, J. M., and Yaksh, T. L., Quantitative assessment of tactile allodynia in the rat paw. J. Neurosci. Methods., 1994; 53: 55-63.
81. Hoch, W., Campanelli, J. T., Harrison, S., and Scheller, R. H., Structural domains of agrin required for clustering of nicotinic acetylcholine receptors. EMBO J., 1994; 13: 2814-2821.
82. MacFarlane, B. V et al., Chronic neuropathic pain and its control by drugs, Pharmacol. Ther., 1997, 75:1-19.
83. Arner S et al., Lack of analgesic effect of opioids on neuropathic and idiopathic forms of pain, Pain, 1988, 39:243-246.
84. Ossipov M. H. et al., Challenges in the development of novel treatment strategies for neuropathic pain, NeuroRx®, 2005, 2:650-661.
85. Nitkin R. M. et al., Identification of agrin, a synaptic organizing protein from Torpedo electric organ, J. Cell Biol., 1987, 105:2471-2478.

The complete disclosures of all references cited in this specification are hereby incorporated by reference. In the event of an otherwise irreconcilable conflict, however, the present specification shall control.

1. A method to ameliorate or decrease neuropathic pain in a mammal, comprising administering to the mammal a therapeutically effective amount of a compound selected from the group consisting of agrin, a C-terminal agrin fragment, and SCP-M1.

2. The method as in claim 1, wherein the compound is a homolog or derivative of agrin.

3. The method as in claim 1, wherein the C-terminal agrin fragment is selected from the group of fragments with sizes of 25 kDa, 50 kDa, 70 kDa, and 90 kDa.

4. The method as in claim 1, wherein the compound is a homolog or derivative of a C-terminal agrin fragment.

5. A method to prevent neuropathic pain due to surgery or injury in a mammal, comprising administering to the mammal at the time of surgery or injury a therapeutically effective amount of a compound selected from the group consisting of agrin, a C-terminal agrin fragment, SCP-1, and SCP-M1.

6. The method as in claim 5, wherein the compound is a homolog or derivative of agrin.

7. The method as in claim 5, wherein the C-terminal agrin fragment is selected from the group of fragments with sizes of 25 kDa, 50 kDa, 70 kDa, and 90 kDa.

8. The method as in claim 5, wherein the compound is a homolog or derivative of a C-terminal agrin fragment.

9. A method to prevent neuropathic pain due to surgery or injury in a mammal due to the decrease in agrin concentration, comprising administering to the mammal within three days after the surgery or injury a therapeutically effective amount of a compound selected from the group consisting of agrin, a C-terminal agrin fragment, SCP-1, and SCP-M1.

10. The method as in claim 9, wherein the compound is a homolog or derivative of agrin.

11. The method as in claim 9, wherein the C-terminal agrin fragment is selected from the group of fragments with sizes of 25 kDa, 50 kDa, 70 kDa, and 90 kDa.

12. The method as in claim 9, wherein the compound is a homolog or derivative of a C-terminal agrin fragment.

13. The method as in claim 9, wherein the compound is administered within one day after the surgery or injury.

14. A method to increase the agrin concentration in the brain, comprising administering to the subject a therapeutically effective amount of a compound selected from the group consisting of SCP-1, and SCP-M1.

15. A method for identifying a substance for its ability to alleviate neuropathic pain, comprising:
a. Identifying a substance that upregulates the expression of agrin in neuronal cells expressing mammalian agrin by administering a candidate substance to said cells in vitro; and
b. Assaying for the ability of said substance to increase the agrin expression in vitro, wherein the ability of said substance to increase the agrin expression in vitro is indicative of a substance which alleviates neuropathic pain.

16. A method for identifying a substance for its ability to alleviate neuropathic pain, comprising:
a. Identifying a substance that increases the ability of agrin to increase the phosphorylation of NMDA receptor subunit NR1 in neuronal cells by administering a candidate substance to said cells in vitro; and
b. Assaying for the amount of phosphorylation of NMDA receptor subunit NR1, wherein the ability of said substance to increase the phosphorylation of NMDA receptor subunit NR1 is indicative of a substance which alleviates neuropathic pain.


SEQUENCE LISTING (12 sequences)

SEQ ID NO: 1 - DNA, 22 bp, Artificial sequence
  Synthetic primer
tggcagtgacggtgttgactac

SEQ ID NO: 2 - DNA, 19 bp, Artificial sequence
  Synthetic primer
cacggcgggacaggcatac

SEQ ID NO: 3 - DNA, 22 bp, Artificial sequence
  Synthetic primer
tcaggagcaaagagcccatagc

SEQ ID NO: 4 - DNA, 22 bp, Artificial sequence
  Synthetic primer
atgtaggtccgcccatcaaagg

SEQ ID NO: 5 - DNA, 25 bp, Artificial sequence
  Synthetic primer
aaccagtctcgcaacatcaatcttc

SEQ ID NO: 6 - DNA, 23 bp, Artificial sequence
  Synthetic primer
gcctcctcatctccactcagttc

SEQ ID NO: 7 - DNA, 19 bp, Artificial sequence
  Synthetic primer
cccaccctccgagcctacc

SEQ ID NO: 8 - DNA, 21 bp, Artificial sequence
  Synthetic primer
gcccatccaacagagccagag

SEQ ID NO: 9 - DNA, 21 bp, Artificial sequence
  Synthetic primer
ccuaguguugaggauccagtt

SEQ ID NO: 10 - DNA, 21 bp, Artificial sequence
  Synthetic primer
cuggauccucaacacuaggtt

SEQ ID NO: 11 - DNA, 21 bp, Artificial sequence
  Synthetic primer
gcccucaaaguccugugautt

SEQ ID NO: 12 - DNA, 21 bp, Artificial sequence
  Synthetic primer
aucacaggacuuugagggctt